(19) United States
(12) Patent Application Publication
(10) Pub. No.: US 2007/0184452 A1
(43) Pub. Date: Aug. 9, 2007

(54) Identification of a gene and mutation responsible for autosomal recessive congenital hydrocephalus

(75) Inventors: Michael L. Robinson, Pataskala, OH (US); Brian E. Davy, Columbus, OH (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: CHILDREN'S HOSPITAL INC, Columbus, OH (US)

(21) Appl. No.: 10/650,449

(22) Filed: Aug. 27, 2003

Related U.S. Application Data
(60) Provisional application No. 60/406,285, filed on Aug. 27, 2002.
(60) Provisional application No. 60/485,440, filed on Jul. 8, 2003.

(51) Int. Cl. C12Q 1/68; C07H 21/04; C12P 21/06; C07K 14/705

(52) U.S. Cl. 435/6; 435/69.1; 435/320.1; 435/325; 530/350; 536/23.5

(57) ABSTRACT

The invention relates to the polynucleotide sequence of the Hydrocephalus-associated gene (Hydin), the polypeptide it encodes and uses therefore. The invention also relates to the mutation in the Hydin gene that is responsible for the development of hydrocephalus. The invention provides for methods of diagnosing hydrocephalus and cilia dysfunction-related disorders.

### RELATED APPLICATIONS

The present application claims priority to U.S. Provisional Application No. 60/406,285 filed Aug. 27, 2002 and U.S. Provisional Application No. 60/485,440 filed Jul. 8, 2003. All of the above-identified applications are incorporated herein by reference in their entirety.

### FIELD OF INVENTION

The invention relates to the polynucleotide sequence of a Hydrocephalus-associated gene (Hydin), the polypeptide it encodes and uses therefore. The invention also relates to the mutation in the Hydin gene that is responsible for the development of hydrocephalus.

### BACKGROUND

Hydrocephalus is an abnormal accumulation of cerebrospinal fluid (CSF) within cavities, known as ventricles, in the brain. As the CSF accumulates within the brain, it causes the ventricles to enlarge and the pressure inside the head to increase. Hydrocephalus is a complex disorder which can result from a variety of different congenital malformations or acquired conditions. Congenital hydrocephalus occurs in approximately 1 in every 1000 births.

### Categories and Treatment of Hydrocephalus

As a consequence of normal development, all parts of the central nervous system surround a continuous lumen originating from the neural tube. This lumen is expanded within the brain to form the lateral, third and fourth ventricles. Clusters of capillaries, surrounded by ependymal cells, line the floor of the lateral ventricles and the roof of the third and fourth ventricles. This capillary network is known as the choroid plexus. It is within the choroids plexus that cerebrospinal fluid (CSF) is produced by a combination of filtration and active transport processes. The central nervous system is entirely surrounded by CSF. CSF serves to protect, nourish and remove waste products from the brain. CSF is continuously produced and circulated through the brain's ventricular system. CSF produced in the lateral ventricles flows into the third ventricle through the interventricular foramen, also known as the foranen of Monro. The CSF flows from the third ventricle into the fourth ventricle through the long, narrow cerebral aqueduct, also known as the aqueduct of Sylvius. While a small portion of the CSF flows from the fourth ventricle into the continuous apertures (foramina of Luschka) and the median appeture (foramen of Magendie) into the subarachnoid space surrounding the brain and spinal cord. CSF is normally reabsorbed into the bloodstream through durul sinuses surrounding the arachnoid villi. The human adult choroid plexus produces approximately 500 ml of CSF every day, which is enough to completely fill the entire fluid spaces of the central nervous system four times over. Therefore, production and reabsorption of CSF is a continuous process in the normal brain.

Hydrocephalus results when there is excess CSF in the ventricular system of the central nervous system. Hydrocephalus can be congenital or acquired. Congenital hydrocephalus results from errors in the development or function of the ventricular system that are present at birth. Congenital hydrocephalus is further subdivided into syndromal (being part of a constellation of typical malformations such as Dandy-Walker Syndrome) or non-syndromal (isolated hydrocephalus). Acquired hydrocephalus results from non-genetic causes, such as tumors or injuries that interfere with normal production or circulation of CSF after birth. Hydrocephalus can be caused by a blockage of CSF flow through the subarachnoid space. Non-communicating or obstructive hydrocephalus results from a blockage in the flow of CSF through the ventricles. The long narrow structure of the cerebral aqueduct between the third and fourth ventricles makes this structure a particularly common site of CSF blockage. The hydrocephalus resulting from the overproduction or under-absorption of CSF is known as communicating hydrocephalus, because there is no blockage in the interventricular flow of CSF.

As hydrocephalus progresses, the ventricles tend to enlarge, and the intracranial pressure increases. In infants and young children, the expansion of the ventricles and the increase in the intracranial pressure results in enlargement of the head. In older children and adults, the closed cranial sutures prevent enlargement of the head, and ventricular dilation is accompanied by compression and thinning of the brain tissue. (Heimer, The Human Brain and Spinal Cord: Functional Neuroanatomy and Dissection Guide. 1983, New York; Springer-Verlag). While there are individual variations or normal ventricular volume, untreated progressive hydrocephalus invariably results in brain damage and eventual death. (Schurr and Polkey Hydrocephalus: Oxford Medical Publications. 21993, Oxford University Press: New York, N.Y.) Postnatal progressive hydrocephalus is most commonly treated by the surgical insertion of a shunt into the ventricular system. (James, Am Fam. Physician, 45: 773-42, 1992) These shunts commonly drain excess CSF into the peritoneal cavity (shunt). While shunting procedures have dramatically improved the prognosis of progressive hydrocephalus, CSF shunts require life-long maintenance and are prone to potentially lethal complications. (Casey, Pediatr. Neurosurg., 27: 63-70, 1997; Epstein, Clin. Neurosurg. 32: 608-631, 1985) Several attempts have been made to treat progressive congenital hydrocephalus detected in utero with fetal surgery using ventriculoamniotic shunts. Despite occasional success with this procedure (Goldstein et al., Fetal Diag. Ther., 5: 84-91, 1990), the long term prognosis of fetuses diagnosed with progressive congenital hydrocephalus remains poor. Many of the fetuses that survive to birth die during the first year of life, or suffer from severe mental and physical handicaps (Pretorius et al., AJR Am. J. Roentgenol. 144: 827-31, 1985)

### Genetics of Human Hydrocephalus

It is currently thought that a significant portion of human congenital hydrocephalus is genetic in origin, although the molecular genetics of human hydrocephalus remains poorly understood.

L1CAM is a neuronal cell adhesion molecule, belonging to the immunoglobulin superfamily, mainly expressed on neurons and schwann cells. (Fransen et al., Hum. Mol. Genet. 6: 1625-32, 1997) The phenotypes associated with L1CAM mutations are variable. L1CAM is known to be responsible for a wide spectrum of neurological abnormalities, many of which were thought to be distinct clinical entities. The pathological cause of L1CAM associated hydrocephalus is stenosis of the cerebral aqueduct causing obstructive, non-communicating hydrocephalus. Recently, male mice deficient in L1CAM expression developed many of the phenotypes associated with human mutations in L1CAM including spastic paraplegia and hydrocephalus. (Dahme et al., Nature Genetics, 17: 346-9, 1997) The hydrocephalus phenotype in these mice was dependent on the strain background, indicating that modifier genes are capable of influencing the L1CAM mutant phenotype. This explains much of the phenotypic variability often seen in human families among relatives with the same L1CAM mutations. (Schrander-Stumpel et al., Am. J. Med Genet., 57: 107-16, 1995; Fransen et al., Am. J. Med Genet, 64: 73-7, 1996) While mutations in the L1CAM gene are responsible for many, if not most, cases of human X-linked hydrocephalus, there is abundant evidence that autosomal recessive forms of human hydrocephalus exist. X-linked hydrocephalus overwhelmingly affects males and is thought to occur with a frequency of 1 in 30,000 male births. (Donnai et al., Eur. J. Pediatr. Surg., 3(suppl. 1): 5-7, 1993)

Autosomal recessive hydrocephalus would be expected to affect equal number of males and females. Several cases of multiple female or mixed sex siblings having hydrocephalus have been reported where autosomal recessive hydrocephalus was suspected. Some of these involve obstructive hydrocephalus associated with the cerebral aqueduct (Castro-Gago et al., Childs Nerv. Syst. 12: 188-91, 1996), the third ventricle (Chow et al., Am. J. Med Genet, 35: 310-3, 1990), the foramen of Monro (Chudley et al., Am. J. Med Genet., 68: 350-6, 1997) or the foramina of the fourth ventricle (Teebi et al., Am. J. Med Genet, 31: 467-70, 1988). In other cases, no obstructions were obvious and the hydrocephalus appeared to be communicating. (Game et al., Am. J. Med Genet, 33: 276-9, 1989) Particularly interesting is a group of Palestinian Arab families where autosomal recessive hydrocephalus appears to be relatively common. (Zlotagoto et al., Am. J. Med Genet, 49:202-4, 1994; Zlotogota et al., Am. J. Med Genet, 71: 33-5, 1997) Despite several reports of human hydrocephalus following an autosomal recessive mode of inheritance, the disease genes or chromosomal locations associated with these cases are entirely unknown.

### Mouse Models of Congenital Hydrocephalus

Mice have proven to be exceptional models for many human genetic diseases. In addition, to being easily maintained and propagated, the genetic information available for mice is unparalleled by any non-human vertebrate. Furthermore, the conservation of gene order in genomic segments identified between mouse and human chromosomes makes it possible to reasonably predict the human chromosomal location of any given gene based on its position in the mouse genome.

Several distinct autosomal recessive mutations leading to hydrocephalus have been reported in mice (Bronson and Lane, Brain Res. Dev. Brain Res., 54: 131-4, 1990; Clark, Proc. Natl. Acad. USA, 18: 654-656, 1932; Dickie, Mouse News Lett., 39: 27, 1968; Falconer and Sierts-Roth, Z. Indukt. Abstamm Verebungsl, 84: 71-73, 1951; Gruneberg, J. Genet., 45: 1-21, 1943, Gruneberg, J. Genet, 45: 22-28, 1943; Hollander, Iowa State J. Res. 51:13-23 1976; Punt et al., J. Neurol. Neurosurg. Psychiatry, 45: 280, 1982; Zimmermann, Z. Indukt. Abstamm Verebungsl 64: 176-180, 1933). Recently the transcription factors Foxc1 (Mf1) and Lmx1a were identified as responsible for the hydrocephalus mutations congenital hydrocephalus (ch) (Kume et al., Cell, 93: 985-96, 1998), and dreher (dr) (Millonig et al, Nature 403: 764-9, 2000), respectively. The genes responsible for the mutations hydrocephalus 1-3 (hy1, hy2 and hy3), hydrocephalus with hop gait (hyh), obstructive hydrocephalus (oh) and hop-sterile (hop) remain unidentified. In addition to these spontaneous mouse mutations, hydrocephalus is at least part of the phenotype in several engineered mice created by both insertional mutations (McNeish et al., J. Exp. Zool., 253: 151-62, 1990) and targeted mutations (Dahme et al., Nat. Genetics 17: 346-9, 1997; das Neves et al., Proc. Natl. Acad. Sci. USA, 96: 11946-51, 1999; Fransen et al. Hum. Mol. Genet., 7: 999-1009, 1998; Homanics et al. Proc. Natl. Acad. Sci. USA 90: 2389-93, 1993; Huang et al. J. Clin. Invest. 96: 2152-61, 1995; Ibanez-Tallon et al., Hum. Mol. Genet. 11:715-21, 2002; Lindeman et al., Genes Dev. 12: 1092-8, 1998).

There are several mouse models for genetic hydrocephalus where the approximate chromosomal position of the mutation has been mapped. In some of these mutants, hydrocephalus represents a small portion of a complex phenotype, for example legless. (McNeish et al., J. Exp. Zool. 253: 151-62, 1990) In other cases, hydrocephalus is the primary phenotype. Mouse hydrocephalus mutations association with specific chromosomal location are shown below in Table 1. In addition to these listed mutants, there are additional autosomal recessive mouse hydrocephalus mutations that have not been mapped, including obstructive hydrocephalus and SUM/NP. Of these mutations, only congenital hydrocephalus (ch) and dreher (dr) have has been cloned.

TABLE 1
Mouse Hydrocephalus Mutations

| Mutation | Chromosome | Inheritance | Reference |
|---|---|---|---|
| Congenital | 13 | Autosomal | Gruneberg, J. Genet., |
| hydrocephalus | | recessive | 45: 22-28, 1943 |
| dreher | 1 | Autosomal | Falconer et al., Z. Indukkt |
| | | recessive | Abstamm |
| | | | Veresbungs, 84: 71-73, |
| | | | 1951 |
| hop-sterile | 6 | Autosomal | Hollander, Iowa State |
| | | recessive | J. Res. 51: 13-23, |
| | | | 1976 |
| hydrocephalus | 8 | Autosomal | Gruneberg J. Genet., |
| 3 | | recessive | 45: 22-28, 1943 |
| hydrocephaly | 7 | Autosomal | Bronson et al., Brain |
| with hop gait | | recessive | Res. Dev. Brain Res. |
| | | | 54: 131-6. 1990 |
| legless | 12 | Autosomal | McNeish et al., J. |
| | | recessive | Exp. Zool. 253: 151-62, |
| | | | 1990 |

Targeted disruption of the winged helix/forkhead transcription factor gene Mf1 (Foxc1) resulted in a phenotype indistinguishable from ch/ch homozygotes. Subsequently, the ch mutation was found to be a stop codon in the Mf1 forkhead domain. (Kume et al., 1998, supra.) Human patients, heterozygous for mutations in FKHL7/freac3 (a presumed homolog of Mf1/Foxc1) suffer from abnormalities in the anterior chamber of the eye including, congenital glaucoma, Axenfeld-Rieger anomaly, and iridogoniodsgenesis. Heterozygous ch mice exhibit similar ocular abnormalities. No human patients with homozygous mutations in FKHL/freac3 have been confirmed in the literature. Hydrocephalus has been described in patients with deletions encompassing the region on chromosome six where FKHL/freac3 is mapped (Davies et al., Hum. Genet., 98: 454-9, 1996), and one family with multiple siblings exhibiting Axenfeld-Rieger anomaly and hydrocephalus has been described. (Moog et al., Am. J. Med. Genet., 33: 276-279, 1989)

Two new hydrocephalus genes were inadvertently discovered by gene targeting. One of these is the murine nuclear factor-1 (Nfia) gene on mouse chromosome 4. (das Neves et al., 1999, supra.) The other involves the disruption of the transcription factor E2F5 leading to the overproduction of CSF in homozygous mutant mice. (Lindeman et al., I genes Dev. 12: 1092-8, 1998) The E2F5 gene has been mapped to chromosome 3 in mice, and has been mapped to human chromosome 8. Additionally, transgenic mice overexpressing TGFβ-1 in the central nervous system develop hydrocephalus. (Galbreath et al., J. Neuropathol. Exp. Neurol., 54: 339-49, 1995, Wyss-Coroy et al., Am. J. Pathol., 147: 53-67, 1995) The intrathecal injection of recombinant TGFβ-1 had previously been shown to induce communicating hydrocephalus in mice (Tada et al., J. Neuroimmunol., 50: 153-8, 1994) and elevated levels of TGFβ-1 have been reported in human communicating hydrocephalus following subacrachnoid hemorrhydine. (Kitazawa et al., Stroke, 25: 1400-4, 1994). The development of hydrocephalus in these cases may be related to an increased production of extracellular matrix within the subarachnoid space leading to decreased CSF reaborsption. (Wyss-Coroy et al., 1995, supra.) Mice are used to model human genetic disease because, as illustrated by the ch heterozygotes, a mutation in a given gene will often produce a similar phenotype in mouse and man. (Melton, Bioassays, 16: 633-8, 1994) Therefore, it is reasonable to believe that most, if not all, of the mouse hydrocephalus mutations will have a clinical human counterpart.

### hy3 Mutation

The hy3 mutation was first identified by Hans Gruneberg, and described as an autosomal recessive inherited phenotype including nasal discharge, runting and hydrocephalus with variable postnatal onset and survival time (Gruneberg 1943b, supra). R. J. Berry further characterized the hy3 mutant phenotype after several generations of inbreeding and reported a consistent runted phenotype in presumed homozygotes with accompanying hydrocephalus, but no nasal discharge (Berry, J. Path. Bact. 81: 157-161, 1961) Berry also reported that the incidence of hydrocephalus among offspring of heterozygous hy3 parents as 16.6%, significantly less than the expected 25% for an autosomal recessive trait with complete penetrance. (Berry, 1961, supra.) The inbred mutant stock was obtained and maintained as heterozygotes (stock HyIII/Le) by the Jackson Laboratory until 1995 when the original inbred stock stopped breeding, and the mutation was then recovered and maintained on a mixed C57BL/6 and CBA/Ca hybrid background. No molecular markers have been reported to identify heterozygous versus homozygous mutant mice, and therefore, hy3 heterozygotes are traditionally identified by test mating. We observed a frequency of hydrocephalus among offspring of heterozygous hy3 parents of 22.3%. This is higher than the 16.6% observed by Berry and much closer to the expected 25% for a fully penetrant autosomal recessive phenotype.

The hy3 mutation was originally mapped by three point test cross by Margaret C. Green and communicated to Roy Robinson of the Jackson Laboratory by a personal letter in 1970 (Jeff Ceci, personal communication). In this letter, Dr. Green placed hy3 approximately 17 cM distal to Os (oligosyndactylism) and 11 cM proximal to e (recessive yellow) on mouse chromosome 8. This information was used to place the location of hy3 at 57 cM on the consensus chromosome 8 linkage map (Blake et al. Nucl. Acids Res. 30: 113-5, 2002). Using molecular markers it was determined that the transgene insertion site does not recombine with D8Mit151 in 188 backcross animals in the combined BSB and BSS interspecific backcross mapping panel from the Jackson Laboratory. (Fryns et al., Ann Genet., 24: 124-5, 1981; Naritomi et al., Clin. Genet., 33: 372-5, 1988; Rivera et al., Clin. Genet., 20: 465-99, 1985).

Current methods for pre-natal diagnosing of autosomal recessive congenital hydrocephalus are not optimally reliable, provide false negatives and may not be adequately informative at early points during pregnancy.

### SUMMARY OF INVENTION

A new transgene-induced insertional mutation, OVE459, resulting in autosomal recessive hydrocephalus is described herein. This mutation does not complement the spontaneous mutation hy3 and represents a new allele of this gene. Furthermore we report genetic and physical mapping of the transgene insertion locus, and cloning of the Hydrocephalus-associated gene (Hydin) that contains a frame shift mutation in hy3/hy3 mice.

The Hydin gene and protein described herein are identical to the Hag gene and protein disclosed in U.S. Provisional Application Nos. 60/406,285 and 60/485,440.

The present invention provides for the identification and characterization of the novel murine Hydin gene and corresponding human homolog sequences. The murine Hydin cDNA sequence is set out as SEQ ID NO: 1 and is 15769 bases and encompasses 87 exons. The murine Hydin gene is located on chromosome 8 and is an allele of the hy3 gene that was previously known in the art. In homozygous hy3/hy3 mice, which spontaneously generate congenital hydrocephalus, there is a frame shift mutation within exon 15 that results in a stop codon. This mutation is believed to be responsible for autosomal recessive congenital hydrocephalus. The predicted human Hydin cDNA sequence is set out as SEQ ID NO: 14. The human Hydin cDNA sequence is 15.7 kB (15783 bases). The predicted human Hydin polypeptide sequence is set out as SEq ID NO: 15.

The present invention provides for the polynucleotide sequence of the murine Hydin gene set out as SEQ ID NO: 1, the genomic sequence of the murine Hydin gene set out as SEQ ID NO: 3 or SEQ ID NO: 16, the predicted human Hydin gene set out as SEQ ID NO: 14. The present invention also provides for polynucleotides which encode the full length polypeptide sequence of SEQ ID NO: 2, SEQ ID NO: 15 or fragments thereof. The invention provides for polynucleotides that hybridize under stringent conditions to (a) the complement of the nucleotides sequence of SEQ ID NOS: 1, 3, 14 or 16; (b) a polynucleotide encoding the polypeptide of SEQ ID NOS: 2 or 15; (c) a polynucleotide which is an allelic variant of any polynucleotides recited above; (d) a polynucleotide which encodes a species homolog of any of the proteins recited above; or (e) a polynucleotide that encodes a polypeptide comprising a specific domain or truncation of the polypeptides of SEQ ID NOS: 2 or 15.

The present invention also provides genes corresponding to the cDNA sequences disclosed herein. The corresponding genes can be isolated in accordance with known methods using the sequence information disclosed herein. Such methods include the preparation of probes or primers from the disclosed sequence information for identification and/or amplification of genes in appropriate genomic libraries or other sources of genomic materials. Further 5′ and 3′ sequence can be obtained using methods known in the art. For example, full length cDNA or genomic DNA that corresponds to the polynucleotide of SEQ ID NOS: 1, 3, 14 or 16 can be obtained by screening appropriate cDNA or genomic DNA libraries under suitable hybridization conditions using the polynucleotides of SEQ ID NOS: 1, 3, 14 or 16 or a portion thereof as a probe. Alternatively, the polynucleotide of SEQ ID NOS: 1, 3, 14 or 16 may be used as the basis for suitable primer(s) that allow identification and/or amplification of genes in appropriate genomic DNA or cDNA libraries.

The nucleic acid sequences of the invention can be assembled from ESTs and sequences (including cDNA and genomic sequences) obtained from one or more public databases, such as dbEST, gbpri, and UniGene, and commercially-available private databases. The EST sequences can provide identifying sequence information, representative fragment or segment information, or novel segment information for the full-length gene.

The polynucleotides of the invention also provide polynucleotides including nucleotide sequences that are substantially equivalent to the polynucleotides recited above. Polynucleotides according to the invention can have, e.g., at least about 65%, at least about 70%, at least about 75%, at least about 80%, 81%, 82%, 83%, 84%, 85%, 86%, 87%, 88%, or 89%, more typically at least about 90%, 91%, 92%, 93%, or 94% and even more typically at least about 95%, 96%, 97%, 98% or 99% sequence identity to a polynucleotide recited above.

Included within the scope of the nucleic acid sequences of the invention are nucleic acid sequence fragments that hybridize under stringent conditions to the nucleotide sequences of SEQ ID NOS: 1, 3, 14 or 16 or complements thereof, which fragment is greater than about 5 nucleotides, preferably 7 nucleotides, more preferably greater than 9 nucleotides and most preferably greater than 17 nucleotides. Fragments of, e.g. 15, 17, or 20 nucleotides or more that are selective for (i.e., specifically hybridize to any one of the polynucleotides of the invention) are contemplated. Probes capable of specifically hybridizing to a polynucleotide can differentiate polynucleotide sequences of the invention from other polynucleotide sequences in the same family of genes or can differentiate murine genes from genes of other species, and are preferably based on unique nucleotide sequences.

The term “stringent” is used to refer to conditions that are commonly understood in the art as stringent. Stringent conditions can include highly stringent conditions (i.e., hybridization to filter-bound DNA under in 0.5 M NaHPO₄, 7% sodium dodecyl sulfate (SDS), 1 mM EDTA at 65° C., and washing in 0.1×SSC/0.1% SDS at 68° C.), and moderately stringent conditions (i.e., washing in 0.2×SSC/0.1% SDS at 42° C.). In instances wherein hybridization of deoxyoligonucleotides is concerned, additional exemplary stringent hybridization conditions include washing in 6×SSC/0.05% sodium pyrophosphate at 37° C. (for 14-base oligos), 48° C. (for 17-base oligos), 55° C. (for 20-base oligos), and 60° C. (for 23-base oligos).

The sequences falling within the scope of the present invention are not limited to these specific sequences, but also include allelic and species variations thereof. Allelic and species variations can be routinely determined by comparing the sequence provided in SEQ ID NOS: 1, 3, 14 or 16, a representative fragment thereof, or a nucleotide sequence at least 90% identical, preferably 95% identical, to SEQ ID NOS: 1, 3, 14 or 16 with a sequence from another isolate of the same species. Furthermore, to accommodate codon variability, the invention includes nucleic acid molecules coding for the same amino acid sequences as do the specific open reading frames (ORF) disclosed herein. In other words, in the coding region of an ORF, substitution of one codon for another codon that encodes the same amino acid is expressly contemplated.

Species homologs (or orthologs), in particular human homologs, of the disclosed polynucleotides and proteins are also provided by the present invention. Species homologs may be isolated and identified by making suitable probes or primers from the sequences provided herein and screening a suitable nucleic acid source from the desired species. The following human BAC clones have been identified within the human chromosome 16 contig represented by the Accession No. NT 010635 to share consensus sequences with the mouse Hydin gene and are contemplated to be portions of the human Hydin homolog: BAC CTA-427H10 (Accession No. AC130459), BAC RP11-240C17 (Accession No. AC109135), BAC RP11-424M24 (Accession No. AC027281), and BAC CIT987SK-A-911E12 (Accession NO. AC003964).

The isolated polypeptides of the invention include, but are not limited to, a polypeptide comprising: the amino acid sequences set forth as any one of SEQ ID NOS: 2 or 15 or an amino acid sequence encoded by the nucleotide sequences SEQ ID NOS: 1, 3, 14 or 16 or the corresponding full length or mature protein. Polypeptides of the invention also include polypeptides preferably with biological or immunological activity that are encoded by: (a) a polynucleotide having the nucleotide sequences set forth in SEQ ID NOS: 1, 3, 14 or 16 or (b) polynucleotides encoding the amino acid sequence set forth as SEQ ID NOS: 2 or 15 or (c) polynucleotides that hybridize to the complement of the polynucleotides of either (a) or (b) under stringent hybridization conditions. The invention also provides biologically active or immunologically active variants of the amino acid sequences set forth as SEQ ID NOS: 2 or 15 or the corresponding full length or mature protein; and “substantial equivalents” thereof (e.g., with at least about 65%, at least about 70%, at least about 75%, at least about 80%, at least about 85%, 86%, 87%, 88%, 89%, at least about 90%, 91%, 92%, 93%, 94%, typically at least about 95%, 96%, 97%, more typically at least about 98%, or most typically at least about 99% amino acid identity) that retain biological activity. Polypeptides encoded by allelic variants may have a similar, increased, or decreased activity compared to polypeptides comprising SEQ ID NOS: 2 or 15. The present invention also provides for compositions comprising the polypeptides described above and a carrier.

Fragments of the proteins of the present invention which are capable of exhibiting biological activity are also encompassed by the present invention. Fragments of the protein may be in linear form or they may be cyclized using known methods, for example, as described in. Saragovi, et al., Bio/Technology 10: 773-778, 1992 and in McDowell, et al., J. Amer. Chem. Soc. 114: 9245-9253, 1992, both of which are incorporated herein by reference. Such fragments may be fused to carrier molecules such as immunoglobulins for many purposes, including increasing the valency of protein binding sites.

Protein compositions of the present invention may further comprise an acceptable carrier, such as a hydrophilic, e.g., pharmaceutically acceptable, carrier.

The present invention further provides isolated polypeptides encoded by the nucleic acid fragments of the present invention or by degenerate variants of the nucleic acid fragments of the present invention. By “degenerate variant” is intended nucleotide fragments which differ from a nucleic acid fragment of the present invention (e.g., an ORF) by nucleotide sequence but, due to the degeneracy of the genetic code, encode an identical polypeptide sequence. Preferred nucleic acid fragments of the present invention are the ORFs that encode proteins.

### Frame Shift Mutation in the Hydin in hy3 Mice

The invention provides for a frame shift mutation in exon 15 of the Hydin gene which results in a premature stop codon and truncated transcription of the gene. This frame shift mutation spontaneously occurs in homozygous hy3 mutant mice and therefore the frame shift mutation is contemplated to be responsible for the initiation of hydrocephalus in the hy3 mutant mice.

The polynucleotides of the invention are useful for detection of the frame shift mutation or any other mutation that plays a role in initiating or progressing hydrocephalus. The Hydin gene and fragments thereof, in particular fragments comprising exons 16 through 87 of the Hydin gene, are useful for screening for heterozygous carriers of the frame shift mutation or any other hydrocephalus related mutation in the Hydin gene.

Current methods for pre-natal diagnosis of autosomal recessive congenital hydrocephalus include ultrasound techniques, DNA linkage analysis in a chronic villous biopsy and cytogenetic studies. Even though diagnostic methods are available, there is no definitive proof of autosomal recessive hydrocephalus; and the existing tests are not optimally reliable and provide false negative. In addition, the ultrasound methods may not detect congenital hydrocephalus until the second trimester of pregnancy and that point it may too late to seek fetal therapy or termination of the pregnancy. Therefore, the identification of additional markers for autosomal recessive congenital hydrocephalus will make screening methods more reliable. In addition, markers which identify heterozygous carriers of the autosomal recessive gene responsible for congenital hydrocephalus identify high-risk parents.

The present invention further provides methods to identify the presence, mutation or expression of the Hydin gene, or homolog thereof, in a test sample, using a nucleic acid probe or antibodies of the present invention, optionally conjugated or otherwise associated with a suitable label.

In one embodiment, the invention provides for methods of detecting the Hydin gene comprising the steps of contacting a biological sample with a compound that binds to a Hydin polynucleotide and detecting binding between the compound and the polynucleotide, wherein binding indicates the presence of the Hydin gene in the sample. In another embodiment, the invention provides for methods of detecting the Hydin polypeptide of the invention comprising the steps of contacting a biological sample with a compound that binds to a Hydin polypeptide and detecting binding between the compound and the polypeptide, wherein binding indicates the presence of the Hydin gene in the sample. The invention also provides methods of detecting a mutation in the human Hydin gene comprising steps of contacting a biological sample with a compound that binds to a Hydin polynucleotide and detecting binding between the compound and the polynucleotide, wherein binding indicates the presence of a mutation in the human Hydin gene in the sample. The mutations detected include a mutation at a position that corresponds to the OVE459 mutation in the murine Hydin gene.

In general, methods for detecting the Hydin gene or mutations thereof can comprise contacting a sample with a compound that binds to and forms a complex with the polynucleotide for a period sufficient to form the complex, and detecting the complex, so that if a complex is detected, a polynucleotide of the invention is detected in the sample. Such methods can also comprise contacting a sample under stringent hybridization conditions with nucleic acid primers that anneal to a polynucleotide of the invention under such conditions, and amplifying annealed polynucleotides, so that if a polynucleotide is amplified, a polynucleotide of the invention is detected in the sample.

In general, methods for detecting a polypeptide of the invention can comprise contacting a sample with a compound that binds to and forms a complex with the polypeptide for a period sufficient to form the complex, and detecting the complex, so that if a complex is detected, a polypeptide of the invention is detected in the sample.

In detail, such methods comprise incubating a test sample with one or more of the antibodies or one or more of the nucleic acid probes of the present invention and assaying for binding of the nucleic acid probes or antibodies to components within the test sample.

The invention provides for methods of diagnosing hydrocephalus or a ciliary dysfunction-related disorder comprising detecting a mutation in the Hydin gene by contacting a biological sample with a compound that binds to the Hydin polynucleotide having the nucleic acid sequence of SEQ ID NO: 1, SEQ ID NO: 3, SEQ ID NO: 14 or SEQ ID NO: 16 and detecting binding between the compound and the polynucleotide, wherein detection of the mutation is indicative of hydrocephalus or a ciliary dysfunction-related disorder.

Conditions for incubating a nucleic acid probe or antibody with a test sample vary. Incubation conditions depend on the format employed in the assay, the detection methods employed, and the type and nature of the nucleic acid probe or antibody used in the assay. One skilled in the art will recognize that any one of the commonly available hybridization, amplification or immunological assay formats can readily be adapted to employ the nucleic acid probes or antibodies of the present invention. Examples of such assays can be found in Chard, T., An Introduction to Radioimmunoassay and Related Techniques, Elsevier Science Publishers, Amsterdam, The Netherlands (1986); Bullock, G. R. et al., Techniques in Immunocytochemistry, Academic Press, Orlando, Fla. Vol. 1 (1982), Vol. 2 (1983), Vol. 3 (1985); Tijssen, P., Practice and Theory of Immunoassays: Laboratory Techniques in Biochemistry and Molecular Biology, Elsevier Science Publishers, Amsterdam, The Netherlands (1985). The test samples of the present invention include cells, protein or membrane extracts of cells, or biological fluids such as cerebral spinal fluid, amniotic fluid, sputum, blood, serum, plasma, or urine. The test sample used in the above-described method will vary based on the assay format, nature of the detection method and the tissues, cells or extracts used as the sample to be assayed. Methods for preparing protein extracts or membrane extracts of cells are well known in the art and can be readily be adapted in order to obtain a sample which is compatible with the system utilized.

The invention also provides for polypeptide-specific nucleic acid hybridization probes capable of hybridizing with naturally occurring nucleotide sequences. The hybridization probes of the subject invention may be derived from the nucleotide sequence SEQ ID NOS: 1, 3, 14 or 16. Because the Hydin gene is only expressed in a limited number of tissues, a hybridization probe derived from of the nucleotide sequence of SEQ ID NOS: 1, 3, 14 or 16 can be used as an indicator of the presence of RNA of cell type of such a tissue in a sample. Any suitable hybridization technique can be employed, such as, for example, in situ hybridization. PCR as described in U.S. Pat. Nos. 4,683,195 and 4,965,188 provides additional uses for oligonucleotides based upon the nucleotide sequences. Such probes used in PCR may be of recombinant origin, may be chemically synthesized, or a mixture of both. The probe will comprise a discrete nucleotide sequence for the detection of identical sequences or a degenerate pool of possible sequences for identification of closely related genomic sequences. Other means for producing specific hybridization probes for nucleic acids include the cloning of nucleic acid sequences into vectors for the production of mRNA probes. Such vectors are known in the art and are commercially available and may be used to synthesize RNA probes in vitro by means of the addition of the appropriate RNA polymerase as T3, T7 or SP6 RNA polymerase and the appropriate radioactively labeled nucleotides. The nucleotide sequences may be used to construct hybridization probes for mapping their respective genomic sequences. The nucleotide sequence provided herein may be mapped to a chromosome or specific regions of a chromosome using well-known genetic and/or chromosomal mapping techniques. These techniques include in situ hybridization, linkage analysis against known chromosomal markers, hybridization screening with libraries or flow-sorted chromosomal preparations specific to known chromosomes, and the like. The technique of fluorescent in situ hybridization of chromosome spreads has been described, among other places, in Verma et al (1988) Human Chromosomes: A Manual of Basic Techniques, Pergamon Press, New York N.Y.

Fluorescent in situ hybridization of chromosomal preparations and other physical chromosome mapping techniques may be correlated with additional genetic map data. Correlation between the location of a nucleic acid on a physical chromosomal map and a specific disease (or predisposition to a specific disease) may help delimit the region of DNA associated with that genetic disease. The nucleotide sequences of the subject invention may be used to detect differences in gene sequences between normal, carrier or affected individuals.

### Connection with Cilia Function

Experimental and clinical studies have linked cilia dysfunction and/or absence of cilia expression to hydrocephalus. Homozygous mutant mice, which have a disruption in the winged helix factor hepatocyte nuclear factor gene, have a complete absence of cilia and exhibit hydrocephalus at about 1 week of age. (Chen et al., J. Clin. Invest. 102(6): 1077-1082, 1998). Mice with mutations in the Polaris gene (Tg737) have decreased cilia in the lining of the ventricular lumen and also exhibited hydrocephalus. (Taulman et al., Mol. Cell. Biol. 12: 589-599, 2001). This relationship is substantiated by the report that a golden retriever diagnosed with primary ciliary dyskinesia, was additionally diagnosed with hydrocephalus after necropsy. (Reichler et al., J. Small Anim. Pract., 42: 345-8, 2001). In addition, members of a large Joranian family with recurrent pulmonary infections were diagnosed with both primary ciliary dyskinesia and hydrocephalus. (Mayo Clin. Proc. 76(12): 1219-24, 2001).

Scanning electron microscopic studies have determined the hy3 mice with severe hydrocephalus at day 18 and 35 had a progressive decrease in cilia populations as the roof of the ventricles were approached. The cilia populations ranged from normal at the basal regions to complete absence at the roof of the ventricle. In addition, the cilia that were presence within the ventricle of the 35 day hy3 mouse were short and had an abnormal appearance. (Bannister and Mundy, Acta Neurochir, 46: 159-168, 1979).

The expression pattern of the Hydin transcript in the fetal and adult mouse suggests that Hydin expression and cilia function may be related. As describe below in Example 6, the Hydin transcript is highly expressed in the following ciliated tissues: chroroid plexus, ependymal cells, the respiratory tract and the testes. In addition the Hydin gene is known to be expressed in the following organisms which have motive cilia: (amphibians) Xenopus tropicalis, Xenopus laevis, (mammals) Mus musculus domesticus, Rattus norvegicus, Bos taurus, Sus scrofa, (fish) Danio rerio, Fagu rubripes, (sea squirts) Ciona intestinalis, Ciona savignyi and (single cell photosynthetic algae) Chlamydomonas reinhardtii. However, Hydin gene expression was undetectable in C. elegans which do not possess motile cilia. Therefore, mutations in the Hydin gene, such as the frame shift mutation described herein, are contemplated to play a role in ciliary dysfunction-related disorders. The Hydin polynucleotide sequence of SEQ ID NOS: 1, 3, 14 or 16 or fragments thereof may be effective diagnostic indicators of ciliary dysfunction-related disorders. The administration of the Hydin polypeptide may effectively treat or prevent ciliary dysfunction-related disorders. The congenital disorders may be treated in utero with gene therapy based on the Hydin gene or polypeptide. The ciliary dysfunction-related disorders in addition to hydrocephalus, contemplated by the present invention include, but are not limited to, Kartagener syndrome, primary ciliary dyskinesia, chronic respiratory diseases such as chronic sinusitis and chronic rhinitis, male infertility, deafness and kidney failure.

The invention provides for methods of diagnosing a ciliary dysfunction-related disorder comprising detecting a mutation in the Hydin gene by contacting a biological sample with a compound that binds to the polynucleotide having the nucleic acid sequence of SEQ ID NO: 14 and detecting binding between the compound and the polynucleotide, wherein detection of the mutation is indicative of a ciliary dysfunction disorder.

### Gene Therapy

Mutations in the Hydin gene may result in loss of normal function of the encoded protein. The invention thus provides gene therapy to restore normal activity of the polypeptides of the invention; or to treat disease states involving polypeptides of the invention. Delivery of a functional gene encoding polypeptides of the invention to appropriate cells is effected ex vivo, in situ, or in vivo by use of vectors, and more particularly viral vectors (e.g., adenovirus, adeno-associated virus, or a retrovirus), or ex vivo by use of physical DNA transfer methods (e.g., liposomes or chemical treatments). See, for example, Anderson, Nature 392(supp.): 25-20 (1998). For additional reviews of gene therapy technology see Friedmann, Science, 244. 1275-1281 (1989); Verma, Scientific American: 68-84 (1990); and Miller, Nature, 357: 455-460 (1992). Introduction of any one of the nucleotides of the present invention or a gene encoding the polypeptides of the present invention can also be accomplished with extrachromosomal substrates (transient expression) or artificial chromosomes (stable expression).

The present invention still further provides cells genetically engineered in vivo to express the polynucleotides of the invention, wherein such polynucleotides are in operative association with a regulatory sequence heterologous to the host cell which drives expression of the polynucleotides in the cell. These methods can be used to increase or decrease the expression of the polynucleotides of the present invention.

Knowledge of DNA sequences provided by the invention allows for modification of cells to permit, increase, or decrease, expression of endogenous polypeptide. Cells can be modified (e.g. by homologous recombination) to provide increased polypeptide expression by replacing, in whole or in part, the naturally occurring promoter with all or part of a heterologous promoter so that the cells express the protein at higher levels. The heterologous promoter is inserted in such a manner that it is operatively linked to the desired protein encoding sequences. See, for example, PCT International Publication No. WO 94/12650, PCT International Publication No. WO 92/20808, and PCT International Publication No. WO 91/09955. It is also contemplated that, in addition to heterologous promoter DNA, amplifiable marker DNA (e.g., ada, dhfr, and the multifunctional CAD gene which encodes carbamyl phosphate synthase, aspartate transcarbamylase, and dihydroorotase) and/or intron DNA may be inserted along with the heterologous promoter DNA. If linked to the desired protein coding sequence, amplification of the marker DNA by standard selection methods results in co-amplification of the desired protein coding sequences in the cells.

In another embodiment of the present invention, cells and tissues may be engineered to express an endogenous gene comprising the polynucleotides of the invention under the control of inducible regulatory elements, in which case the regulatory sequences of the endogenous gene may be replaced by homologous recombination. As described herein, gene targeting can be used to replace a gene's existing regulatory region with a regulatory sequence isolated from a different gene or a novel regulatory sequence synthesized by genetic engineering methods. Such regulatory sequences may be comprised of promoters, enhancers, scaffold-attachment regions, negative regulatory elements, transcriptional initiation sites, regulatory protein binding sites or combinations of said sequences. Alternatively, sequences which affect the structure or stability of the RNA or protein produced may be replaced, removed, added, or otherwise modified by targeting. These sequences include polyadenylation signals, mRNA stability elements, splice sites, leader sequences for enhancing or modifying transport or secretion properties of the protein, or other sequences which alter or improve the function or stability of protein or RNA molecules.

The targeting event may be a simple insertion of the regulatory sequence, placing the gene under the control of the new regulatory sequence, e.g., inserting a new promoter or enhancer or both upstream of a gene. Alternatively, the targeting event may be a simple deletion of a regulatory element, such as the deletion of a tissue-specific negative regulatory element. Alternatively, the targeting event may replace an existing element; for example, a tissue-specific enhancer can be replaced by an enhancer that has broader or different cell-type specificity than the naturally occurring elements. Here, the naturally occurring sequences are deleted and new sequences are added. In all cases, the identification of the targeting event may be facilitated by the use of one or more selectable marker genes that are contiguous with the targeting DNA, allowing for the selection of cells in which the exogenous DNA has integrated into the cell genome. The identification of the targeting event may also be facilitated by the use of one or more marker genes exhibiting the property of negative selection, such that the negatively selectable marker is linked to the exogenous DNA, but configured such that the negatively selectable marker flanks the targeting sequence, and such that a correct homologous recombination event with sequences in the host cell genome does not result in the stable integration of the negatively selectable marker. Markers useful for this purpose include the Herpes Simplex Virus thymidine kinase (TK) gene or the bacterial xanthine-guanine phosphoribosyl-transferase (gpt) gene.

The gene targeting or gene activation techniques which can be used in accordance with this aspect of the invention are more particularly described in U.S. Pat. No. 5,272,071 to Chappel; U.S. Pat. No. 5,578,461 to Sherwin et al.; International Application No. PCT/US92/09627 (WO93/09222) by Selden et al.; and International Application No. PCT/US90/06436 (WO91/06667) by Skoultchi et al., each of which is incorporated by reference herein in its entirety.

### Compositions

Pharmaceutical compositions comprising the Hydin polypeptide of the invention are provided. The pharmaceutical compositions may comprise one or more additional ingredients such as pharmaceutically effective carriers. Dosage and frequency of the administration of the pharmaceutical compositions are determined by standard techniques and depend, for example, on the weight and age of the individual, the route of administration, and the severity of symptoms. Administration of the pharmaceutical compositions may be by routes standard in the art, for example, parenteral, intravenous, oral, buccal, nasal, pulmonary, rectal, or vaginal.

### Antibodies

The present invention provides for antibodies and antibody fragments that bind to the Hydin polypeptide. The antibodies may be polyclonal including monospecific polyclonal, monoclonal (mAbs), recombinant, chimeric, humanized such as CDR-grafted, human, single chain, and/or bispecific, as well as fragments, variants or derivatives thereof. Antibody fragments include those portions of the antibody which bind to an epitope on the Hydin polypeptide. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions.

Polyclonal antibodies directed toward the Hydin polypeptide generally are produced in animals (e.g., rabbits or mice) by means of multiple subcutaneous or intraperitoneal injections of the Hydin polypeptide and an adjuvant. It may be useful to conjugate a Hydin polypeptide or fragment thereof to a carrier protein that is immunogenic in the species to be immunized, such as keyhole limpet heocyanin, serum, albumin, bovine thyroglobulin, or soybean trypsin inhibitor. Also, aggregating agents such as alum are used to enhance the immune response. After immunization, the animals are bled and the serum is assayed for Hydin antibody titer.

Monoclonal antibodies directed toward Hydin polypeptide are produced using any method which provides for the production of antibody molecules by continuous cell lines in culture. Examples of suitable methods for preparing monoclonal antibodies include the hybridoma methods of Kohler et al., Nature, 256:495-497 (1975) and the human B-cell hybridoma method, Kozbor, J. Immunol., 133:3001 (1984); Brodeur et al., Monoclonal Antibody Production Techniques and Applications, pp. 51-63 (Marcel Dekker, Inc., New York, 1987). Also provided by the invention are hybridoma cell lines which produce monoclonal antibodies reactive with Hydin polypeptides.

Antibodies which specifically bind to the Hydin polypeptide may be used to provide reagents for use in diagnostic assays for the detection of the Hydin polypeptide in various body fluids. In another embodiment, the Hydin polypeptide or fragments thereof may be used as antigens in immunoassays for the detection of Hydin polypeptide in various patient tissues and body fluids including, but not limited to: ambiotic fluid, blood, serum, ear fluid, spinal fluid, sputum, urine, lymphatic fluid and cerebrospinal fluid. The antigens of the present invention may be used in any immunoassay system known in the art including, but not limited to: radioimmunoassays, ELISA assays, sandwich assays, precipitin reactions, gel diffusion precipitin reactions, immunodiffusion assays, agglutination assays, fluorescent immunoassays, protein A immunoassays and immunoelectrophoresis assays.

For diagnostic applications, antibodies that specifically bind Hydin polypeptide may be labeled with a detectable moiety. The detectable moiety can be any one which is capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin; or an enzyme, such as alkaline phosphatase, β-galactosidase, or horseradish peroxidase (Bayer et al., Meth. Enz., 184:138-163 (1990)).

### BRIEF DESCRIPTION OF DRAWINGS

FIG. 1 depicts the 1,552 bp microinjection construct used to create OVE459 transgenic mice. This construct consisted of the murine αA-crystallin promoter (αA) fused to the human BDNF coding sequence (hBDNF) linked to the 3′ UTR and poly-adenylation signal from the α subunit of the bovine follicle stimulating hormone gene (FSH pA). The coding sequence and 3′ UTR were cloned into the CPV2 vector as a 1,159 bp XbaI fragment, and this transgene-specific XbaI fragment was used as a probe for Southern and FISH analysis. Restriction enzyme site shown are SstII (S), NheI (Nh), BamHI (B), XbaI (X), EcoRI (E) and NotI (N).

FIG. 2 depicts FISH mapping of the OVE459 transgene insertion locus. (A) A portion of a single metaphase spread from a hydrocephalic OVE459 transgenic mouse is shown following G-banding, (B) the same metaphase region after hybridization to the transgene-specific Xba I fragment probe. Hybridization was confined to a single site near the distal end of mouse chromosome 8, arrows. (C) An ideogram of mouse chromosome 8 with an arrow marking the approximate site of the OVE459 transgene insertion between G-bands D2-E1.

FIG. 3 is a haplotype figure combining data from The Jackson BSB and BSS backcrosses showing part of Chromosome 8 with loci linked to D8Mlr1. Loci listed in order with the most proximal at the top. The black boxes represent the C57BL6/JEi allele and the white boxes the SPRET/Ei allele. The number of animals with each haplotype is given at the bottom of each column of boxes. The percent recombination (R) between adjacent loci is given to the right of the figure, with the standard error (SE) for each R.

FIG. 4 is a table summarizing the EST sequences and other published sequences that were used to assemble the Hydin full length cDNA sequence. “ACC#” is the accession number of the identified sequence, “exons represented” corresponds to the exon sequence identified and “DNA homology” is the comparison of the Hydin exon and the public sequence.

FIG. 5 sets out the predicted human Hydin cDNA sequence (SEQ ID NO: 14).

FIG. 6 sets out the predicted human Hydin protein sequence (SEQ ID NO: 15).

FIG. 7 sets out the a murine Hydin cDNA sequence that is extended within its 5′ untranslated region (SEQ ID NO: 16).

### DETAILED DESCRIPTION

The following examples illustrate the invention wherein Example 1 describes production of OVE459 transgenic mice, Example 2 demonstrates that OVE459 mice carry a recessive insertional hydrocephalus-inducing mutation, Example 3 demonstrates that OVE459 is an allele of hy3, Example 4 describes the cloning of the transgene insertion in OVE459 mice, Example 5 describes the genetic and physical mapping of the OVE459 transgenic insertion locus, Example 6 describes the cloning and sequencing of the Hydin cDNA, Example 7 describes the identification of the mutation responsible for hydrocephalus in the hy3 mouse and Example 8 describes the identification of homologs of the Hydin gene, in particular the human Hydin gene.

### Example 1

### Production of OVE459 Transgenic Mice

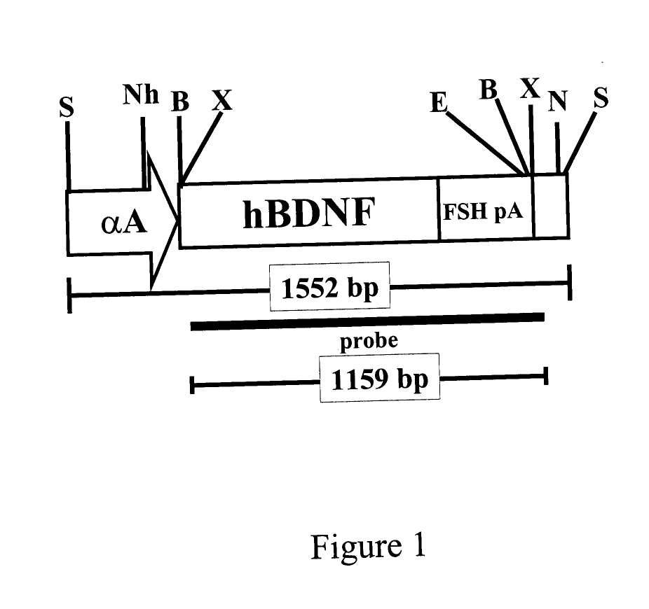

A transgenic construct, αA-BDNF/bFSH, designed to express brain derived neurotropic factor (BDNF) in the developing lens was produced by subcloning a human BDNF cDNA fused to the 3′ UTR of the bovine αFSH gene into the αA-crystallin promoter vector CPV2, replacing the SV40 intron and polyadenylation signal of CPV2. (Robinson et al., Development 121: 505-14, 1995) A schematic of the construct is depicted as FIG. 1. The 1,552 bp microinjection fragment (SEQ ID NO: 4) in pαA-BDNF/bFSH was isolated from the vector by digestion with SStII (Gibco/BRL, Gaithersburg, Md.), purified, and microinjected into FVB/N pronuclear stage mouse embryos as described in Taketo et al. (Proc. Natl. Acad. Sci. USA, 88: 2065-9, 1991).

The microinjection into the mouse zygotes resulted in the production of a single male transgenic founder for the transgenic line designated herein as OVE459. The transgenic founders were identified by PCR analysis using primers PR4 (5′-GCATTCCAGCTGCTGACGGT-3′: SEQ ID NO: 5), a sense primer complimentary to the murine αA-crystallin promoter, and 11421 (5′-ACACCTGGGTAGGCCAAGCCACCTT-3′; SEQ ID NO: 6), an antisense primer complimentary to human BDNF. A diagnostic band of 308 bp was amplified from genomic DNA of transgenic mice following 30 cycles of standard PCR with an annealing temperature of 58° C.

This founder appeared phenotypically normal in all respects including the eye. Three other transgenic founders were produced with a similar transgenic construct CPV2/BDNF differing from αA-BDNF/bFSH only by the replacement of the bovine αFSH 3′ UTR with the SV40 intron and polyadenylation signal of CPV2. These other BDNF transgenic founders also failed to exhibit any phenotypic abnormalities.

The founder for line OVE459 was bred to female FVB/N mice and transmitted the transgene to a portion of his progeny. These F1 hemizygous transgenic mice were phenotypically indistinguishable from their wild type littermates. When hemizygous OVE459 transgenic mice were interbred to produce homozygous transgenic mice, a portion of the pups in the resulting litters usually exhibited a failure to thrive and died, typically between the first and third week after birth. Closer examination revealed that the dying pups could most often be identified by 4-6 days after birth. The most severely affected of these mice died by about 10 days after birth. The less severely affected mice began to exhibit enlargement of the head that progressed until death, typically by 21 days, but always prior to 42 days after birth. Gross necropsy revealed that all affected mice exhibited enlargement of the brain ventricles and characteristic thinning and softening of the top of the skull vault consistent with congenital hydrocephalus. Males and females appear to be equally affected. No such phenotype was ever observed in pups from matings between hemizygous OVE459 mice and wild type mice. These observations led to the hypothesis that the transgenic line OVE459 carried a recessive, transgene-induced insertional mutation leading to congenital hydrocephalus.

Very rarely mice phenotypically typed as heterozygotes at weaning developed lethal hydrocephalus by six weeks of age. No nasal discharge among the homozygous hy3, OVE459 or double heterozygous mice was observed. It is possible that the lack of nasal discharge and increased penetrance of the hydrocephalic phenotype, relative toe that described by Berry (Berry, J. Path. Bact. 81: 157-161, 1961), relates to different sets of modifier loci present in the original and current hy3 stocks.

### Example 2

### OVE459 Mice Carry a Recessive Insertional Hydrocephalus-Inducing Mutation

Southern blot analysis determined that the OVE459 mice carried a recessive insertional hydrocephalus-inducing mutation. Genomic DNA was isolated from hydrocephalic and phenotypically normal OVE459 transgenic mice. Ten micrograms of genomic DNA per lane was digested with EcoRI, BamH I or NheI. Digested DNA was electrophoresed through agarose and transferred to a nylon filter as described in Robinson et al. (Dev. Biol., 198: 13-31, 1998). Blots were probed with the transgene-specific 1,159 bp XbaI fragment (SEQ ID NO: 7 or nucleotides 349-1511 of SEQ ID NO: 4) from pαA-BDNF/bFSH following random-prime labeling with ³²P-dCTP. Washing and exposure to X-ray film was carried out as described Robinson et al. 1998, supra.

Southern blots of genomic DNA from hydrocephalic and phenotypically normal OVE459 transgenic mice hybridized with a transgene-specific probe revealed identical hybridization patterns. Therefore all OVE459 transgenic mice carried the same transgene insertion site and different transgene integration sites could not explain the phenotypic variation among transgenic mice. These analyses also demonstrated that approximately 12-15 copies of the transgene inserted in a single genomic location. In most cases, the production of transgenic mice by pronuclear injection results in transgenic founders containing multiple copies of the transgene in a tandem head-to-tail array at a single genomic locus (Brinster et al. Cell, 27: 223-31, 1981), but more complex integration patterns are possible.

The presence of multiple copies of the transgene within OVE459 transgenic mice facilitated further analysis of this transgenic line by FISH to determine the chromosomal location where the OVE459 transgene inserted. FISH was also used to test the hypothesis that hydrocephalic mice were transgenic homozygotes and that transgenic mice without ventricular enlargement were hemizygous for the transgene.

FISH was carried out as reported in Majumder et al. (Mamm. Genome, 9: 863-868, 1998). Briefly, metaphase chromosome spreads were prepared from both a hydrocephalic and a phenotypically normal OVE459 transgenic mouse. The slides were stained with Giemsa for G-banding and photographed. The slides were then destained and hybridized with a 1,159 bp digoxigenin labeled probe containing the BDNF coding region (SEQ ID NO: 7). Previously photographed G-banded metaphase cells were used to determine the precise location of the transgene insertion. The chromosomes were counter-stained with 0.5 μg/ml propidium iodide in an antifade buffer and the preparations viewed with an Olympus BX60 epifluorescence microscope. Previously photographed G-banded metaphase cells were rephotographed using Kodacolor 100 Gold film.

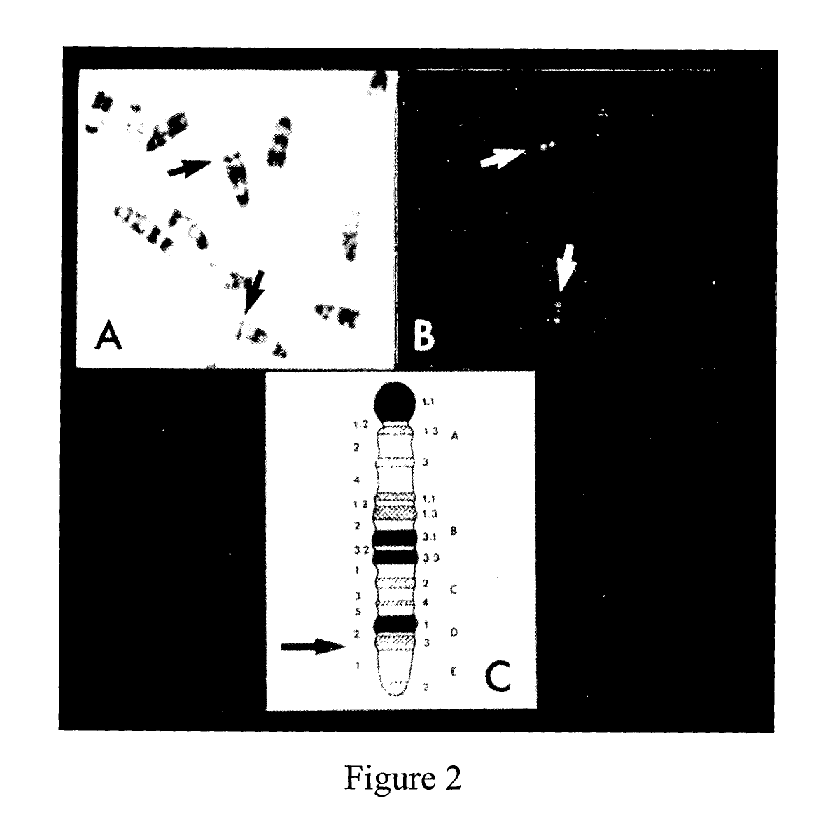

The G-banding and the FISH analysis using the BDNF specific probe are shown in FIG. 2. As predicted, the hydrocephalic mouse was homozygous for the transgene and the non-hydrocephalic mouse was hemizygous for the transgene. FISH also revealed that the transgene array was within a single site near the distal end of mouse chromosome 8 (region D2-E1).

### Example 3

### OVE459 is an Allele of hy3

The spontaneous, recessive, hydrocephalus-inducing mouse mutation hy3 has also been genetically mapped to the distal portion of mouse chromosome 8. The reported phenotype for homozygous hy3 mutant mice closely paralleled the gross abnormalities in homozygous OVE459 transgenic mice. A breeding complementation experiment was carried out to determine if the insertional mutation in OVE459 represented a new allele of hy3. The gene responsible for the mutation in hy3 is unknown and there is currently no published molecular test to distinguish wild type from hy3 mutation carriers in progeny from known heterozygous mutant parents.

Six untested mice (3 males and 3 females) from the B6CBACa-A^{w-J}/A-hy3/+ colony at the Jackson Laboratory (Bar Harbor, Me.), stock number 002703 (the result of matings between two mice proven, by test breeding, to carry the hy3 mutation) were purchased and bred to hemizygous transgenic mice from the OVE459 line. As all homozygous hy3 mice die prior to sexual maturity, each of the six mice purchased from the Jackson Laboratory had a ⅔ probability of carrying the mutation. Therefore, according to the binomial distribution, the probability that none of the six mice carried the mutation was 0.0014. Considering this, we reasoned that if none of the six matings between potential hy3 carriers and transgenic OVE459 mice produced hydrocephalic offspring, the two mutations were unlikely to be allelic.

The resulting pups of untested and hemizygous OVE459 transgenic mice were genotyped for the presence of transgene by PCR and were anesthetized prior to perfusion with PBS followed by Bouin's fixative. Fixed brains were analyzed by gross inspection for signs of ventricular dilation. For determination of hydrocephalic frequency, reported in Table 2, only mating pairs where hydrocephalic pups were born were included for matings including presumed hy3 heterozygotes. Also, only those litters where all pups were accounted for and typed between 10 and 21 days after birth were counted. All OVE459 data are from mice maintained on an FVB/N inbred background. The hy3 mutation has been moved to an FVB/N inbred background since purchase from the Jackson Laboratory. To facilitate this genetic background change, the molecular markers D8Mit248 and D8Mit215 (polymorphic between the original hy3 genetic background and FVB/N) were used to identify likely carriers of the hy3 mutation. D8Mit248 and D8Mit215 are approximately 11 and 5 cM, respectively, on either side of where we believe the hy3 mutation to reside.

Four of these six mating pairs produced hydrocephalic offspring. The carrier status of these four (2 males and 2 females) potential hy3 heterozygotes was subsequently confirmed by interbreeding to obtain hy3/hy3 homozygous pups. Upon gross inspection of the pup's brains, the hydrocephalus in OVE459 homozygotes, hy3/hy3 homozygotes and double OVE459/hy3 heterozygotes was indistinguishable, both in terms of the kinetics and gross pathology. As expected, each of these hydrocephalic offspring, and all subsequent hydrocephalic offspring between OVE459 and hy3 mice, was positive for the transgene. The proportion of hydrocephalic offspring resulting from mating between OVE459 hemizygotes and between OVE459 hemizygous and hy3 heterozygous mice was very similar and approached the predicted 25% Mendelian ratio of an autosomal recessive trait with full phenotypic penetrance, as summarized in Table 2 below. The complete failure of the hy3 mutant allele to complement the OVE459 insertional mutation is the best evidence that these two mutations are allelic and very likely result in disrupted function in the same gene or set of genes on mouse chromosome 8.

TABLE 2
Frequency of Hydrocephalus Among Offspring of OVE459
and hy3 Matings

| Mating | Total Offspring | Hydrocephalic | Percentage |
|---|---|---|---|
| OVE459 × OVE459 | 829 | 199 | 24.0% |
| hy3 × hy3 | 584 | 130 | 22.3% |
| OVE459 × hy3 | 355 | 79 | 22.3% |
| Overall Total | 1768 | 408 | 23.1% |

### Example 4

### Cloning the Transgene Insertion Site of OVE459 Mice

In contrast to the spontaneous hy3 mutation, where no molecular markers are known, the insertion of the transgene in OVE459 mice provided a molecular tag into the genomic locus likely to contain the gene relevant to the hydrocephalic phenotype. Tight linkage of the hydrocephalus-inducing mutation to the OVE459 transgene insertion sight was suggested by the failure of the mutation to segregate away from the transgene in over 20 generations.

To clone the transgene insertion site, a genomic lambda pHydine library was constructed using DNA from homozygous OVE459 hydrocephalic mice. This library was screened using a transgene-specific hybridization probe (see FIG. 1 and SEQ ID NO: 7). Both the FISH data and the genomic Southern blot probed with a transgene-specific probe indicated that the transgene inserted in a single genomic location, and the Southern blot furthermore suggested that 12-15 copies of the transgene inserted in a tandem array. Since the microinjection construct was approximately 1.6 Kb, the tandem transgene array would be expected to be 19.2 to 24 Kb in length. Since the pHydine clones in the library kit used can only incorporate recombinant inserts of 9-23 Kb, it was unlikely that the entire transgene array with a significant length of flanking genomic DNA on both sides would be recovered. Therefore, we expected to recover three types of transgene-containing pHydine clones using the transgene-specific probe. The clones expected included those containing only transgene and those that contained transgene and the flanking genomic DNA on either the centromeric or telomeric side of the insertion site. Conveniently, in the lambda Fix II vector system used to make the library, both SalI and NotI digestion release the genomic insert from the vector arms. NotI, in contrast to SalI, also cuts the transgene construct once. This allowed the total length of the genomic insert as well as estimate the approximate proportion of the genomic insert that consisted of transgene copies to be estimated.

Of the four lambda clones analyzed in detail, two, designated BAA and CAA, were sequenced as follows. The BAA clone is located 5′ of the BDNF transgene while the CAA clone is located 3′ of the transgene. Initial insert end sequence was determined from purified DNA from lambda clones BAA and CAA using T3 and T7 sequencing primers present in the lambda vector pHydine arms. The unique genomic fragment in lambda clones BAA and CAA were subcloned into pBluescript KS- (Stratagene) as NotI fragments. The resultant plasmid clones pB19 and pC5 were approximately 14 and 16 Kb in size, respectively. Sequence was determined from each end of the plasmids using M13 and M13R primers. Template was prepared using Qiagen minipreps. Dye terminator chemistries were employed and sequence determined on an ABI377 automated sequencer. The GPS-1 Genome Priming system (New England Biolabs) was employed to introduce unique primer-binding sites in each construct per the manufacturer's directions. Internal sequence in each plasmid construct was determined using the PrimerS and PrimerN supplied with the kit. Sequence was determined as above using dye terminator chemistries. Non-transposon sequence was removed and the insert sequence assembled using PHRED/PHRAP/CONSED (Ewing and Green, Genome Res., 8: 186-94, 1998; Gordon et al., Genome Res., 8: 195-202, 1998).

The BAA clone was a unique clone consisting of an insert of 18 Kb. Restriction analysis revealed that clone BAA contained approximately five copies of transgene and a mouse genomic insert of 11 Kb. Another lambda clone, BCA contained a genomic insert consisting only of tandemly arranged transgene copies. The two remaining pHydine clones, CAA and DAA, appeared identical in restriction digests, consisting of a genomic insert of 16 Kb, approximately 3 Kb of which was transgene. Further restriction mapping and Southern blotting confirmed that clones DAA and CAA differed substantially from clone BAA, suggesting that these represented opposite sides of the transgene insertion. PHydine clones BAA and CAA were selected for further analysis and were sequenced as described above.

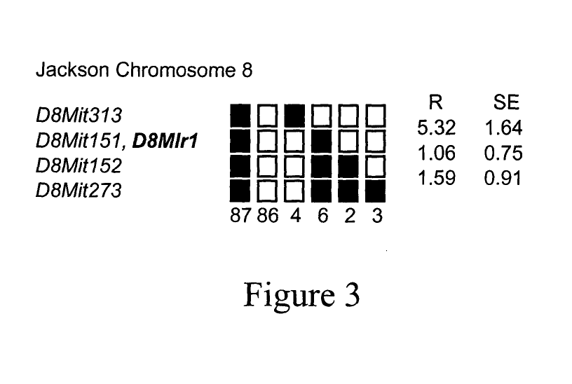

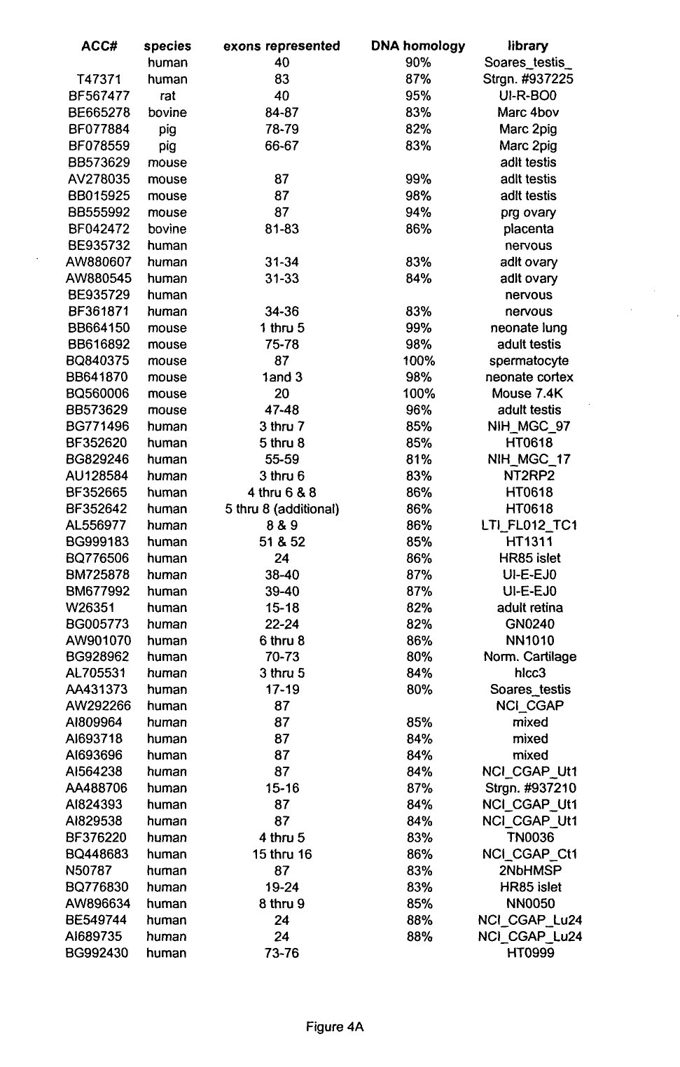

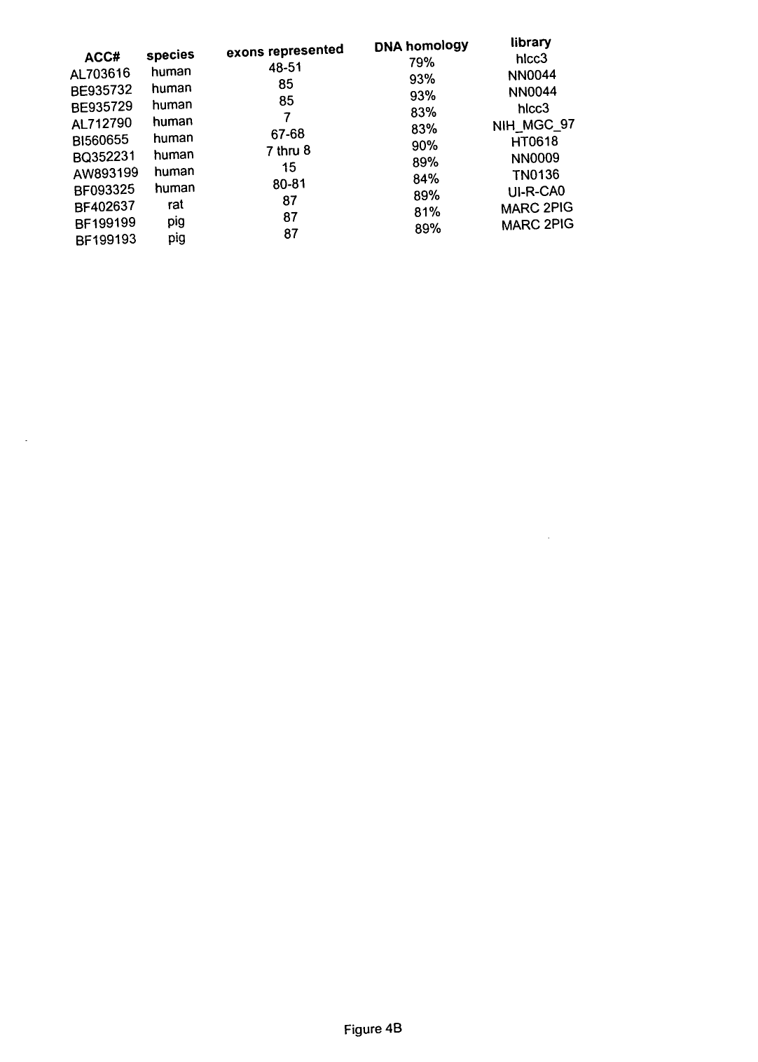

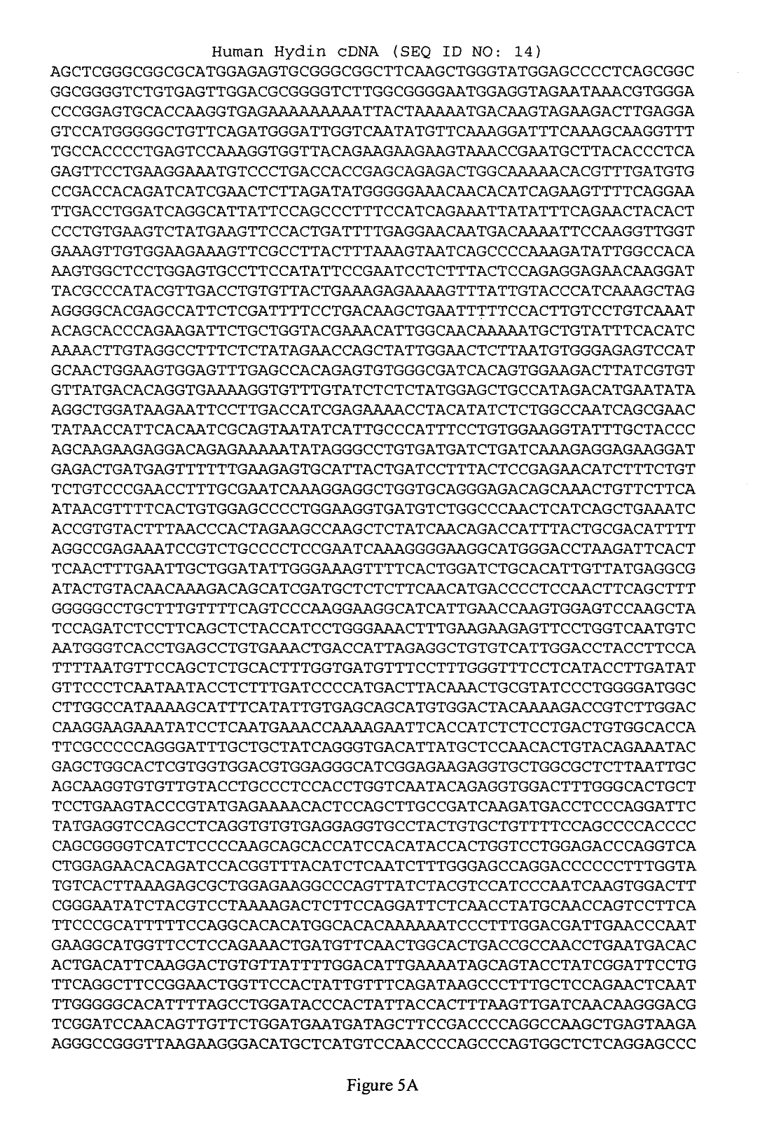

Sequence analysis revealed that the transgene array was on the long (23 Kb) pHydine arm side and the mouse genomic DNA was on the short (9 Kb) pHydine arm side in both pHydine clones as depicted in FIG. 3. The assembly of the BAA and CAA sequences is set out as SEQ ID NO: 8. The transgene is represented as “NNNNNNNNNNNNNNN” (see bases 12088-12102 of SEQ ID NO: 8) and is located between the CAA and BAA sequences.

Unique PCR primer sets were designed to amplify mouse genomic DNA near the distal ends of each pHydine clone, relative to the transgene insertion, using sequence information obtained using the T3 sequencing primer. The primer set derived from the sequence of the mouse genomic insert in pHydine clone CAA approximately 13 Kb from the transgene insert from the genomic region on the pHydine clone CAA clone were designated C1 (5′-CAAAAGAGCTGAGGAAAGATG-3′; SEQ ID NO: 9) and C2 (5′-TAGGATGCAGGGGGTTATT-3′; SEQ ID NO: 10).

The primer set derived from the sequence of the mouse genomic insert in pHydine clone BAA, approximately 11 Kb from the transgene insert, were designated B3 (5′-GGTCCGAGAAAC CTGCCTGCTATCA-3′; SEQ ID NO: 11) and B4 (5′-ACCCACGTCGCCTGTG TTCATTATG-3′; SEQ ID NO: 12). As expected, the PCR primers B3 and B4 from the shorter mouse genomic DNA insert in clone BAA, failed to amplify a band in the longer mouse genomic insert in clone CAA, confirming that these clones represent opposite sides of the transgene insertion.

### Example 5

### Genetic and Physical Mapping of the OVE459 Transgene Insertion Locus

The primer sets B3/B4 (SEQ ID NOS: 11 and 12) and C1/C2 (SEQ ID NOS: 9 and 10) were used to screen C57BL/6JEi and SPRET/Ei genomic DNA for polymorphisms that could be used to map the genetic location of the genomic DNA flanking the transgene insert in OVE459 using the Jackson Laboratory Backcross DNA Panel Mapping Resource as described in Rowe et al., (Mamm. Genome, 5: 253-74, 1994). Genomic DNA from all 188 animals in the combined Jackson BSS (C57BL/6JEi×SPRET/Ei)F1×SPRET/Ei] and BSB [(C57BL/6J×Mus spretus)F1×C57BL/6J] interspecific backcross mapping panels from the Jackson Laboratory Backcross DNA Panel Mapping Resource were genotyped for the PCR polymorphism with primers C1 and C2.

According to the sequence information from the OVE459 genomic clones, the B3/B4 and C1/C2 PCR primers should amplify DNA fragments of 327 bp and 415 bp respectively on FVB/N strain genomic DNA. No polymorphisms were detected using primers B3/B4. In contrast, while the primer set C1/C2 amplified an apparently identically sized band in both FVB/N and C57BL/6JEi DNA, a distinctly larger band was amplified in SPRET/Ei genomic DNA. This polymorphism was used to map the location of the genomic insert in clone CAA in both the BSS and the BSB mapping panel from the Jackson Laboratory consisting of a total of 188 backcross animal (Rowe et al., supra). The C1/C2 polymorphism mapped cleanly in both mapping panels and was assigned the nomen D8M1r1. D8Mlr1 does not recombine with D8Mit151 and was placed 5.32+/−1.64 cM distal to D8Mit313 and 1.06+/−0.75 cM proximal to D8Mit152n as depicted in FIG. 3. This position corresponds to approximately 54 cM on the mouse genome informatics chromosome 8 consensus linkage map as reported in Blake et al. (Nucleic Acids Research, 30: 113-5, 2002).

The interspecific backcross panels provided a precise genetic location for the genomic DNA on the CAA side of the transgene insertion, but as no mapable PCR polymorphism was identified on the opposite flank genetic mapping of the BAA side was not possible. Therefore, we screened a 129/Sv bacterial artificial chromosome (BAC) library (CITB Mouse BAC DNA library pool, Research Genetics, Huntsville, Ala.) using primers B3 and B4 to identify BAC clones that encompassed the OVE459 transgene insertion. BAC DNA clones positive for the B3/B4 primer set were screened with primers C1 and C2 using PCR. Additional BAC clones were identified in the RPCI-23 mouse BAC library by screening filter arrays obtained from Pieter de Jong (Children's Hospital Oakland Research Institute, Oakland, Calif.) by hybridization with the unique genomic inserts from pHydine clones BAA and CAA. The clone RPCI23-21B7 was completely sequenced by the NIH funded Genome Sequencing Network. The accession number for the complete genomic sequence of RPCI23-21B7 is AC069308.

Two BAC clones, 9N1 and 218P4, were positive for the B3/B4 primer set. Of these two clones, 218P4 was also positive for the C1/C2 primer set from the CAA genomic insert, showing that these two amplified regions are physically linked on a single BAC clone. The mouse genomic insert in BAC 218P4 was determined by pulsed field gel electrophoresis to be approximately 120 Kb in size. An additional C57BL/6J BAC clone, RPCI23-21B7, positive for markers on both sides of the transgene insertion site, was identified by hybridization. BAC 21B7 contained a genomic insert of approximately 240 Kb and completely encompassed the genomic region covered by BAC 218P4. From the complete sequence of BAC 21B7, we determined that the markers defined by primer sets B3/B4 and C1/C2 (D8Mlr1) are separated by approximately 51 Kb of wild type genomic DNA, and that the microsatellite markers D8Mit151 and D8Mit213 lie within the intervening sequence.

The sequence information obtained from BAC RPCI23-21B7 indicated that D8Mlr1 is only 47.8 Kb from D8Mit151. While transgene-induced insertional mutations can induce genomic rearrangements making cloning of the relevant genes difficult, these observations suggest the gene responsible for inducing hydrocephalus in hy3 homozygous mice is in close proximity to D8Mit151. Currently, there are no genes or family members of genes that have been previously associated with mammalian hydrocephalus near this region of mouse chromosome 8. The region encompassing D8Mit151 on mouse chromosome 8 corresponds to human chromosome 16q21-23. Haploinsufficiency of this region of 16q has been associated with general growth failure, perinatal death, facial bone dysgenesis, shortened limbs, and hydrocephalus (Fryns et al., Ann Genet., 24: 124-5, 1981; Naritomi et al., Clin. Genet., 33: 372-5, 1988; Rivera et al., Clin. Genet., 20: 465-99, 1985). This analysis provides the most precise location to date for the hydrocephalus-inducing gene in hy3 mutant mice. Hydrocephalus has also been reported in an infant with a balanced translocation t(4;16)(q35;q22.1) including this region (Callen et al. Clin. Genet., 38: 466-8, 1990; Taysi et al., Birth Defect, 14: 343-7, 1978). Furthermore, Sakuragawa and Yokoyama mapped the chromosome 16 breakpoint of this or a similar hydrocephalus-associated t(4;16)(q35;q22.1) translocation between haptoglobin and calretinin (Sakuragawa and Yokoyama, Cong. Anom. 34: 303-310, 1994). This group also discovered a rearrangement of genomic DNA within 1.2 Mb of calretinin using genomic DNA from fibroblasts carrying this translocation. Interestingly, according to the Mouse Genome Sequencing Consortium V3 Assembly, D8Mlr1 is approximately 376 Kb distal to calretinin on mouse chromosome 8.

### Example 6

### Cloning and Sequencing of the Hydin cDNA

BAC clone 218P4 (Research Genetics), described in Example 5, was used in a direct cDNA selection experiment to identify exons on the BAC that were expressed in the wild type neonatal mouse head. Direct cDNA selection technique, as described in Serge et al. (Genomics, 28: 549-59, 1995) and Del Mastro and Lovett (Methods Mol. Biol. 68: 183-199, 1997), allows for the isolation of expressed sequences in a large genomic clone using a complex cDNA pool from a tissue of interest. Briefly, total RNA was isolated from wild type embryonic day 17, newborn and post natal day 2 mouse heads. These developmental stages encompass the time points just prior to the onset of frank hydrocephalus in the OVE459 homozygotes. The RNA was pooled and mRNA was enriched by oligo dT selection, converted to cDNA, digested with restriction enzymes and ligated to linkers. The BAC218P4 was similarly digested and ligated to biotinylated linkers. Repetitive sequencing in the cDNA were blocked by a brief hybridization with Cot1 DNA (Life Technologies) and total DNA from homozygous OVE459 mutant mice. The BAC fragments were then mixed and hybridized to the cDNA fragments at 65° C. for 54 hours. Biotinylated BAC fragments and hybridizing cDNA fragments were isolated by streptavidin coated magnetic beads (Dynal). Selected cDNA's were amplified by PCR and subjected to another round of hybridization and selection prior to blunt end ligation using Novagen's perfectly blunt cloning system. A positive control BAC clone (BAC123), which is known to contain portions of the pola1 and Arx genes, was run in parallel.

This approach identified several exons that were determined to originate from two novel genes that were present, in part on BAC218P4. Subsequently, the identified exons were used to identify additional larger BAC clones encompassing the relevant genomic region. This screen identified RPC123-21B7, which the Trans-NIH Mouse Initiative sequenced at the Applicant's request. The RPCI23-21B7 sequence was assigned Accession No. AC069308. The RPCI23-21B7 complete sequence was used to order the exons discovered in the cDNA selection experiments.

Genomic sequence from human and mouse sequences within public databases and the Celera Genomics database were searched to identify regions of conserved homology in order to predict the location of additional exons. These regions of homology were further examined using splice site prediction software from the Drosophila Genome Project and Genio splice site prediction web site. This software allowed for predictions of the exon boundaries within the transcript. Subsequently, this information was compared to the National Center for Biotechnology Expressed Sequence Tag (EST) data base. The EST sequences identified as segments of the gene are summarized in FIG. 4. The RNA clones identified as segments of the Hydin gene are summarized in Table 3. The EST sequences and the RNA clones identified as segments of Hydin gene exons represent at least part of the corresponding exons.

TABLE 3
RNA Clones Identified As Segments of the Hydin Gene

| | | Exons | Method of | Nucleotide |
|---|---|---|---|---|
| Accession No. | Species | Represented | Prediction | Homology |
| XM_146514 | Mouse | 39-47, 48-80, | Computational | 100% |
| | | 82-87 | annotation | |
| XM_112453 | Mouse | 13-47, 48-80, | Computational | 100% |
| | | 82-87 | annotation | |
| XM_146514 | Mouse | 1, 3-9, 10-13 | Computational | 100% |
| | | | annotation | |
| XM_030075 | Human | 74-87 | Computational | 85% |
| | | | annotation | |
| AL137259 | Human | 75-87 | testis mRNA | 85% |
| | | | clone | |
| AK074472 | Human | 78-87 | lung mRNA | 85% |
| | | | clone | |
| AK02688 | Human | 81-87 | lung mRNA | 87% |
| | | | clone | |
| XM_171789 | Human | 43, 66-69, | Computational | 87% |
| | | 71-80, 81-85 | annotation | |
| NM_017558 | Human | 3-21 | Computational | 87% |
| | | | annotation | |
| BC028351 | Human | 3-16 | testis mRNA | 87% |
| | | | clone | |
| AK02688 | Human | 3-21 | teratocarcinoma | 87% |
| | | | RNA | |
| AB058767 | Human | 35-41, 44-48, | brain mRNA | N/A |
| | | 50-57 | | |
| AL834340 | Human | N/A | brain mRNA | N/A |
| AL833826 | Human | N/A | testis mRNA | N/A |
| | | | clone | |
| AK006604 | Mouse | N/A | testis mRNA | N/A |
| | | | clone | |
| AK016044 | Mouse | N/A | testis mRNA | N/A |
| | | | clone | |
| NM_032821 | Human | N/A | teratocarcinoma | N/A |
| | | | RNA | |
| AK027571 | Human | N/A | teratocarcinoma | N/A |
| | | | RNA | |
| AL122038 | Human | N/A | testis mRNA | N/A |
| | | | clone | |
| AL133042 | Human | N/A | testis mRNA | N/A |
| | | | clone | |
| AL122038 | Human | N/A | testis mRNA | N/A |
| | | | clone | |
| AK057467 | Human | N/A | testis mRNA | N/A |
| | | | clone | |

The identified computer-generated and EST-predicted exons and splice sites were confirmed by amplifying each exon from mouse brain RNA using RT-PCR and sequencing the resulting PCR product. These confirmatory experiments demonstrated that the computer-predicted exons were expressed in mouse brain and linked these predicted exons into a single transcript. TA consensus sequence for the novel gene, denoted herein as Hydin was derived. The full length genomic sequence of Hydin is set out as SEQ ID NO: 3, which contains at least 87 exons spanning approximately 344 kB. The locations of the exons within the genomic sequence are summarized in Table 4.

TABLE 4
Summary of Hydin Exons

| | Position within cDNA | Identified by | | |
|---|---|---|---|---|
| Exon | (SEQ ID | Exon Selection | | RNA clone with |
| Number | NO: 1) | Experiments | EST with Homology | Homology |
| 1 | 1-41 | N/A | BB664150, | XM_146513 |
| | | | BB641870 | |
| 2 | 42-223 | N/A | BB664150 | |
| 3 | 224-381 | N/A | BB664150, | XM_146513, |
| | | | BB641870, | NM_017558, |
| | | | BG771496, | BC028351 |
| | | | AU128584, | |
| | | | AL705531 | |
| 4 | 382-507 | N/A | BB664150, | XM_146513, |
| | | | BG771496, | NM_017558, |
| | | | AU128584, | BC028351 |
| | | | BF352665, | |
| | | | AL705531, | |
| | | | BF376220 | |
| 5 | 508-627 | N/A | BB664150, | XM_146513, |
| | | | BG771496, | NM_017558, |
| | | | AL705531BF352620, | BC028351 |
| | | | AU128584, | |
| | | | BF352665, | |
| | | | BF352642, BF376220 | |
| 6 | 628-762 | N/A | BG771496, | XM_146513, |
| | | | BF352620, | NM_017558, |
| | | | AU128584, | BC028351 |
| | | | BF352665, | |
| | | | BF352642, | |
| | | | AW901070 | |
| 7 | | N/A | BG771496, | XM_146513, |
| | | | BF352620, | NM_017558, |
| | | | BF352642, | BC028351 |
| | | | AW901070, | |
| | | | AL712790, | |
| | | | BQ352231 | |
| 8 | 963-1087 | N/A | BF352620, | XM_146513, |
| | | | BF352665, | NM_017558, |
| | | | BF352642, | BC028351 |
| | | | AL556977, | |
| | | | AW901070, | |
| | | | AW896634, | |
| | | | BQ352231 | |
| 9 | 1088-1289 | N/A | AL556977, | XM_146513, |
| | | | AW896634 | NM_017558, |
| | | | | BC028351 |
| 10 | 1290-1473 | N/A | N/A | XM_146513, |
| | | | | NM_017558, |
| | | | | BC028351 |
| 11 | 1474-1573 | N/A | N/A | XM_146513, |
| | | | | NM_017558, |
| | | | | BC028351 |
| 12 | 1574-1692 | N/A | N/A | XM_146513, |
| | | | | NM_017558, |
| | | | | BC028351 |
| 13 | 1693-1916 | N/A | N/A | XM_112453, |
| | | | | XM_146513, |
| | | | | NM_017558, |
| | | | | BC028351 |
| 14 | 1917-1984 | N/A | N/A | XM_112453, |
| | | | | NM_017558, |
| | | | | BC028351 |
| 15 | 1985-2220 | N/A | W26351, AA488706, | XM_112453, |
| | | | BQ448683, | NM_017558, |
| | | | AW893199 | BC028351 |
| 16 | 2221-2321 | N/A | W2635, AA431373, | XM_112453, |
| | | | BQ448683 | NM_017558, |
| | | | | BC028351 |
| 17 | 2322-2457 | N/A | W2635, AA431373 | XM_112453, |
| | | | | NM_017558 |
| 18 | 2458-2622 | N/A | W2635, AA431373 | XM_112453, |
| | | | | NM_017558 |
| 19 | 2623-2775 | N/A | AA431373, | XM_112453, |
| | | | BQ7768300 | NM_017558 |
| 20 | 2776-3014 | N/A | BQ560006, | XM_112453, |
| | | | BQ776830 | NM_017558 |
| 21 | 3015-3291 | N/A | BQ776830 | XM_112453, |
| | | | | NM_017558 |
| 22 | 3292-3435 | N/A | BG005773, | XM_112453 |
| | | | BQ7768300 | |
| 23 | 3436-3579 | N/A | BG005773, | XM_112453 |
| | | | BQ7768300 | |
| 24 | 3580-3893 | N/A | BQ776506, | XM_112453 |
| | | | BG005773, | |
| | | | BQ776830, | |
| | | | BE549744, AI689735 | |
| 25 | 3894-4031 | N/A | N/A | XM_112453 |
| 26 | 4032-4104 | N/A | N/A | XM_112453 |
| 27 | 4105-4215 | N/A | N/A | XM_112453 |
| 28 | 4216-4425 | N/A | N/A | XM_112453 |
| 29 | 4426-4563 | N/A | N/A | XM_112453 |
| 30 | 4564-4741 | N/A | N/A | XM_112453 |
| 31 | 4742-4869 | N/A | AW880607, | XM_112453 |
| | | | AW880545 | |
| 32 | 4870-5000 | N/A | AW880607, | XM_112453 |
| | | | AW880545 | |
| 33 | 5001-5137 | N/A | AW880607, | XM_112453 |
| | | | AW880545 | |
| 34 | 5138-5265 | N/A | AW880607, | XM_112453 |
| | | | BF361871 | |
| 35 | 5266-5457 | N/A | BF361871 | XM_112453, |
| | | | | AB058767 |
| 36 | 5458-5607 | N/A | BF361871 | XM_112453, |
| | | | | AB058767 |
| 37 | 5608-5847 | N/A | N/A | XM_112453, |
| | | | | AB058767 |
| 38 | 5848-6010 | N/A | BM725878 | XM_112453, |
| | | | | AB058767 |
| 39 | 6011-6184 | N/A | BM725878, | XM_112453, |
| | | | BM677992 | AB058767 |
| 40 | 6185-6358 | N/A | Al126146, BF567477, | XM_146514, |
| | | | BM725878, | XM_112453, |
| | | | BM67799 | AB058767 |
| 41 | 6359-6532 | N/A | N/A | XM_146514, |
| | | | | XM_112453, |
| | | | | AB058767 |
| 42 | 6533-6753 | N/A | N/A | XM_146514, |
| | | | | XM_112453 |
| 43 | 6754-6891 | N/A | N/A | XM_146514, |
| | | | | XM_112453, |
| | | | | XM_171789 |
| 44 | 6892-7075 | N/A | N/A | XM_146514, |
| | | | | XM_112453, |
| | | | | AB058767 |
| 45 | 7076-7217 | N/A | N/A | XM_146514, |
| | | | | XM_112453, |
| | | | | AB058767 |
| 46 | 7218-7386 | N/A | N/A | XM_146514, |
| | | | | XM_112453, |
| | | | | AB058767 |
| 47 | 7387-8004 | N/A | BB57329 | XM_146514, |
| | | | | XM_112453, |
| | | | | AB058767 |
| 48 | 8005-8226 | Exon Selection | BB57329, AL703616 | XM_146514, |
| | | | | XM_112453, |
| | | | | AB058767 |
| 49 | 8227-8435 | Exon Selection | AL703616 | XM_146514, |
| | | | | XM_112453 |
| 50 | 8436-8618 | Exon Selection | AL703616 | XM_146514, |
| | | | | XM_112453 |
| 51 | 8619-8738 | Exon Selection | BG999183, | XM_146514, |
| | | | AL703616 | XM_112453 |
| 52 | 8739-8898 | Exon Selection | BG999183 | XM_146514, |
| | | | | XM_112453 |
| 53 | 8899-9071 | N/A | N/A | XM_146514, |
| | | | | XM_112453 |
| 54 | 9072-9270 | Exon Selection | N/A | XM_146514, |
| | | | | XM_112453 |
| 55 | 9271-9370 | Exon Selection | BG829246 | XM_146514, |
| | | | | XM_112453 |
| 56 | 9371-9470 | N/A | BG829246 | XM_146514, |
| | | | | XM_112453 |
| 57 | 9471-9639 | Exon Selection | BG829246 | XM_146514, |
| | | | | XM_112453 |
| 58 | 9640-9875 | Exon Selection | BG829246 | XM_146514, |
| | | | | XM_112453 |
| 59 | 9876-9993 | Exon Selection | BG829246 | XM_146514, |
| | | | | XM_112453 |
| 60 | 9994-10198 | Exon Selection | N/A | XM_146514, |
| | | | | XM_112453 |
| 61 | 10199-10434 | Exon Selection | N/A | XM_146514, |
| | | | | XM_112453 |
| 62 | 10435-10586 | Exon Selection | N/A | XM_146514, |
| | | | | XM_112453, |
| 63 | 10587-10776 | N/A | N/A | XM_146514, |
| | | | | XM_112453 |
| 64 | 10777-10879 | N/A | N/A | XM_146514, |
| | | | | XM_112453 |
| 65 | 10880-11167 | N/A | N/A | XM_146514, |
| | | | | XM_112453 |
| 66 | 11168-11310 | N/A | BF078559 | XM_146514, |
| | | | | XM_112453, |
| | | | | XM_171789 |
| 67 | 11311-11529 | N/A | BF078559, BI560655 | XM_146514, |
| | | | | XM_112453, |
| | | | | XM_171789 |
| 68 | 11530-11690 | N/A | BI560655 | XM_146514, |
| | | | | XM_112453, |
| | | | | XM_171789 |
| 69 | 11691-11788 | N/A | N/A | XM_146514, |
| | | | | XM_112453, |
| | | | | XM_171789 |
| 70 | 11789-11984 | N/A | BG928962 | XM_146514, |
| | | | | XM_112453, |
| 71 | 11985-12197 | Exon Selection | BG928962 | XM_146514, |
| | | | | XM_112453, |
| | | | | XM_171789 |
| 72 | 12198-12336 | Exon Selection | BG928962, | XM_146514, |
| | | | BG9924300 | XM_112453, |
| | | | | XM_171789 |
| 73 | 12337-12502 | Exon Selection | BG928962, | XM_146514, |
| | | | BG992430 | XM_112453, |
| | | | | XM_171789 |
| 74 | 12503-12650 | Exon Selection | BG992430 | XM_146514, |
| | | | | XM_112453. |
| | | | | XM_030075, |
| | | | | XM_171789 |
| 75 | 12651-12858 | Exon Selection | BB616892 BG992430 | XM_146514, |
| | | | | XM_112453, |
| | | | | XM_030075, |
| | | | | AL137259, |
| | | | | XM_171789 |
| 76 | 12859-13080 | Exon Selection | BB616892, | XM_146514, |
| | | | BG992430 | XM_112453, |
| | | | | XM_030075, |
| | | | | AL137259, |
| | | | | XM_171789 |
| 77 | 13081-13250 | Exon Selection | BB616892 | XM_146514, |
| | | | | XM_112453, |
| | | | | XM_030075, |
| | | | | AL137259, |
| | | | | XM_171789 |
| 78 | 13251-13449 | Exon Selection | BF077884, | XM_146514, |
| | | | BB616892 | XM_112453, |
| | | | | XM_030075, |
| | | | | AL137259, |
| | | | | AK074472, |
| | | | | XM_171789 |
| 79 | 13450-13608 | Exon Selection | BF077884 | XM_146514, |
| | | | | XM_112453, |
| | | | | XM_030075, |
| | | | | AL137259, |
| | | | | AK074472, |
| | | | | XM_171789 |
| 80 | 13609-13886 | Exon Selection | BF093325 | XM_146514, |
| | | | | XM_112453, |
| | | | | XM_030075, |
| | | | | AL137259, |
| | | | | AK074472, |
| | | | | XM_171789 |
| 81 | 13887-14106 | Exon Selection | BF042472, BF093325 | XM_030075, |
| | | | | AL137259, |
| | | | | AK074472, |
| | | | | AK02688, |
| | | | | XM_171789 |
| 82 | 14107-14319 | Exon Selection | BF042472, | XM_146514, |
| | | | | XM_112453, |
| | | | | XM_030075, |
| | | | | AL137259, |
| | | | | AK074472, |
| | | | | AK02688, |
| | | | | XM_171789 |
| 83 | 14320-14483 | Exon Selection | T47371, BF042472 | XM_146514, |
| | | | | XM_112453, |
| | | | | XM_030075, |
| | | | | AL137259, |
| | | | | AK074472, |
| | | | | AK02688, |
| | | | | XM_171789 |
| 84 | 14484-14654 | Exon Selection | BE665278 | XM_146514, |
| | | | | XM_112453, |
| | | | | XM_030075, |
| | | | | AL137259, |
| | | | | AK074472, |
| | | | | AK02688, |
| | | | | XM_171789 |
| 85 | 14635-14865 | Exon Selection | BE665278, | XM_146514, |
| | | | BE935732, | XM_112453, |
| | | | BE935729, | XM_030075, |
| | | | | AL137259, |
| | | | | AK074472, |
| | | | | AK02688, |
| | | | | XM_171789 |
| 86 | 14866-15090 | Exon Selection | BE665278 | XM_146514, |
| | | | | XM_112453, |
| | | | | XM_030075, |
| | | | | AL137259 |
| | | | | AK074472, |
| | | | | AK02688 |
| 87 | 15091-15769 | Exon Selection | BE665278, | XM_146514, |
| | | | AW292266, | XM_112453, |
| | | | AV278035, | XM_030075, |
| | | | BB015925, | AK074472, |
| | | | BB555992, | AL137259, |
| | | | BQ840375, | AK02688 |
| | | | AI809964, AI693718, | |
| | | | AI69396, AI564238, | |
| | | | N50787M, | |
| | | | BF402637, | |
| | | | BF199199, BF199193 | |

The full length murine Hydin cDNA sequence is set out SEQ ID NO: 1 and the murine Hydin genomic sequence is set out as SEQ ID NO: 3. The murine Hydin cDNA encodes an open reading frame of at least 5120 amino acids (SEQ ID NO: 2). The first 58 amino acids are encoded by exon 2, which is an alternately spliced exon that is only present in a subset of Hydin transcripts. The amino acid starting at residue 59 is encoded by exon 3. Exon 1 is predicted to solely contain 5′ untranslated sequences. While it is possible that the Hydin protein has multiple potential initiation codons, is most likely that the methionine at residue 82 is the initiation site since its codon is within the best match for the Kozak consensus for eukaryotic translations. The upstream methionines at positions 75 and 78 are also reasonable matches for the Kozak consensus.

The predicted Hydin polypeptide contains a major sperm protein domain near the amino terminus. Major sperm protein domains are known to be involved in the molecular interactions underlying sperm motility. These domains oligomerise to form an extensive filament system that extends from sperm villipoda, along the leading edge of the pseudopod. Therefore, the Hydin protein is contemplated to be involved in mobility.

In situ hybridization studies indicated Hydin transcripts were expressed in the developing choroids plexus at midgestation of the developing mouse. High transcript expression was detected in certain portions of the ependyma in the newborn mouse brain, and in both upper airways of the lung and throughout the developing spermatids in the testis. Multiple tissue northern blot analysis demonstrated abundant transcript expression only in testes of adult mice.

### Example 7

### Identification of Mutation Responsible for Hydrocephalus

A spontaneous frame shift mutation was identified in exon 15 of the Hydin gene in homozygous hy3 mice. Sequence analysis was carried out on every exon predicted to be within the Hydin gene. This analysis confirmed that the homozygous hy3 mice had a deletion of a single base pair (cytosine) at base 1993 within exon 15. This deletion results in residue 577 and 578 being translated as an isoleucine and proline. This deletion results in a frame shift mutation which generates a stop codon (TGA) at residue 579. Therefore, the frame shift mutation would result in expression of a truncated Hydin protein, which would be missing the majority of the amino acid sequence. It is believed that the identified frame shift mutation is responsible for the development of hydrocephalus.

### Example 8

### Identification of Hydin Homologs

The mouse Hydin gene can be used to search the public database for exons of the human or other organism homologs of the mouse Hydin gene based on EST database searches and evaluation of the mouse human homology in corresponding regions of the respective genomes. For example, the following human BAC clones have been identified to share consensus sequences with the mouse Hydin gene described herein: BAC CTA-427H10 (Acc. No. AC130459), BAC RP11-240C17 (Acc. No. AC109135), BAC RP11-424M24 (Acc. No. AC027281) and BAC CIT987SK-A-911E12 (Acc. No. AC003964).

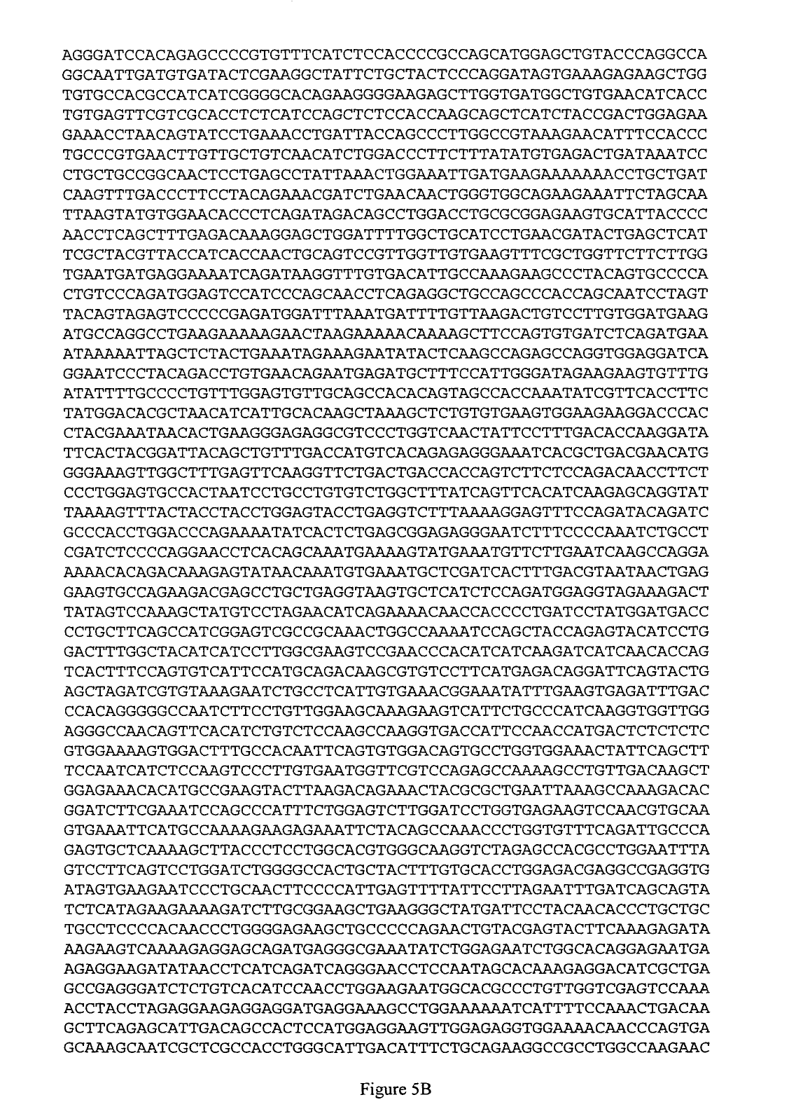

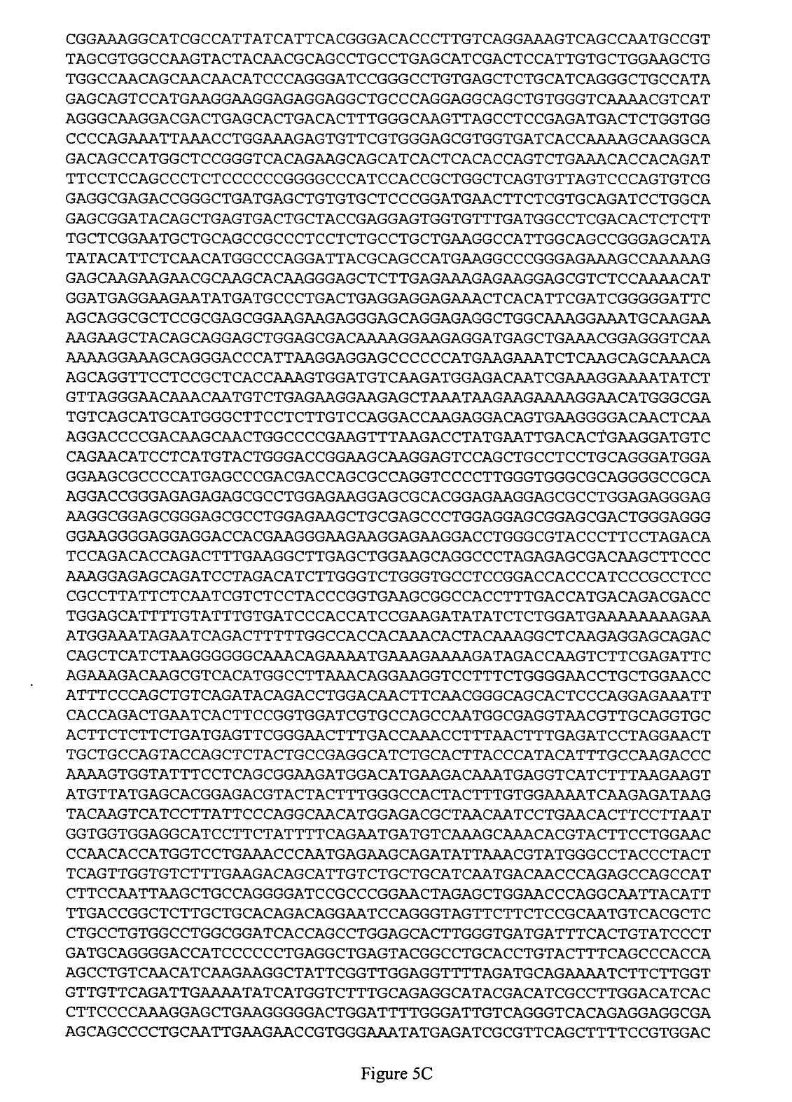

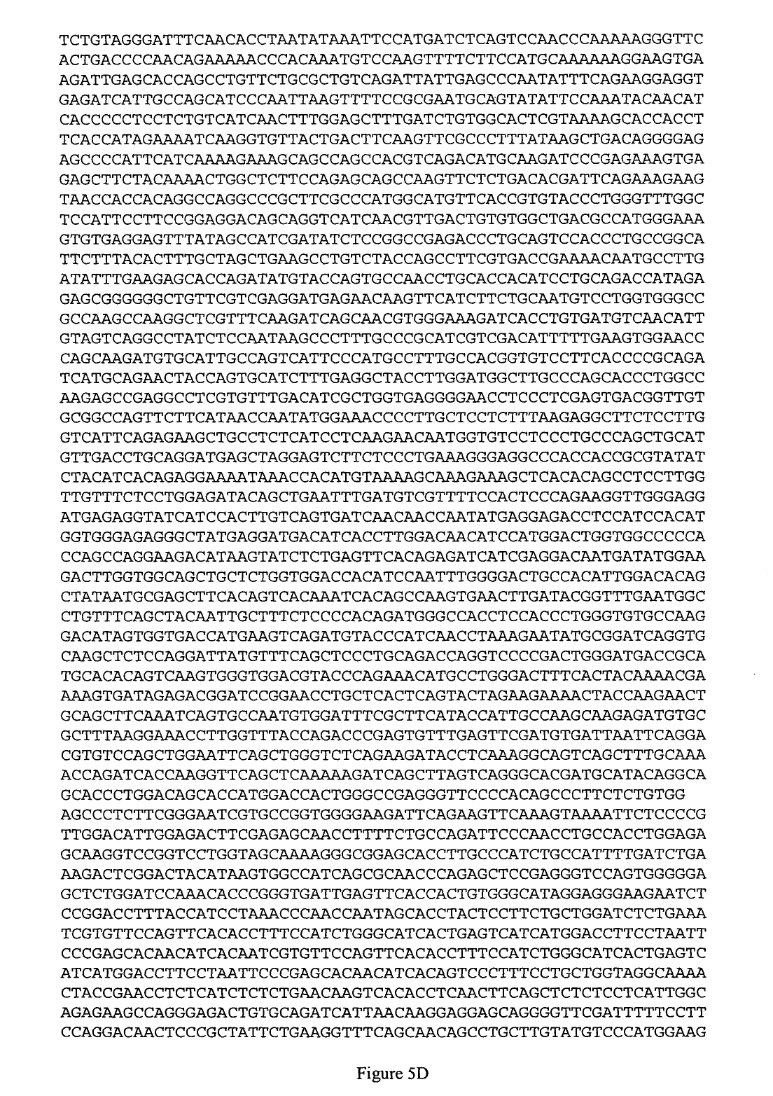

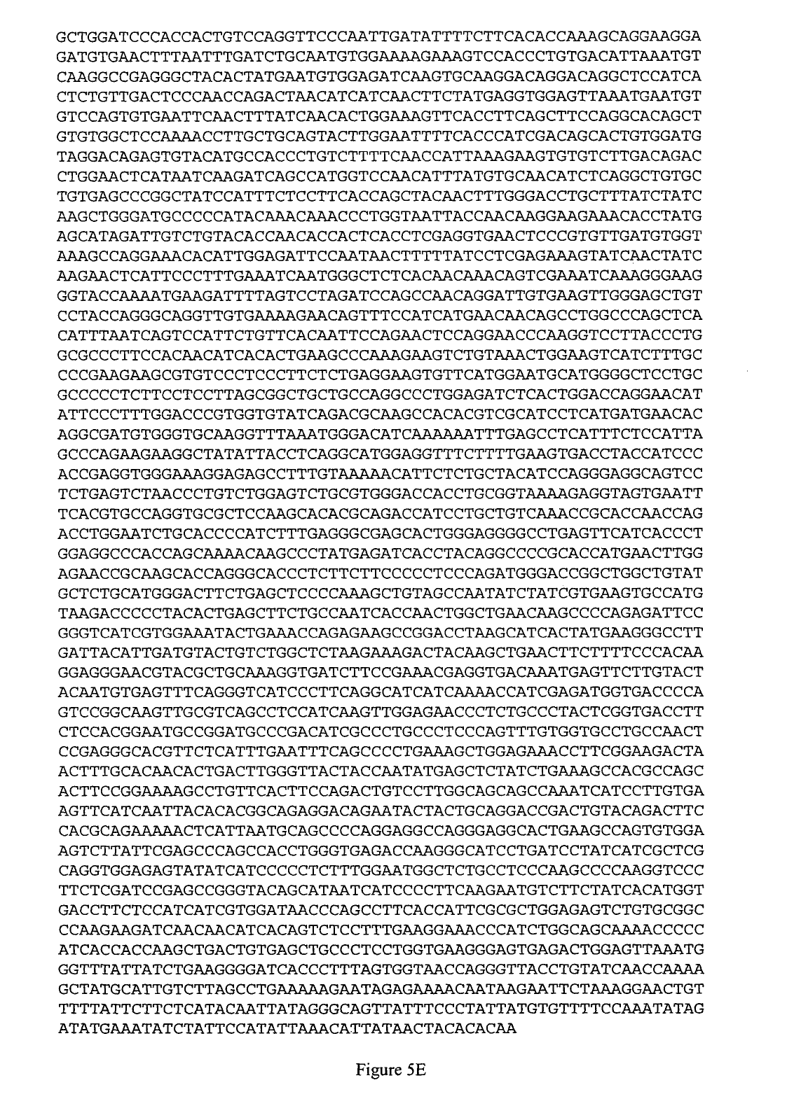

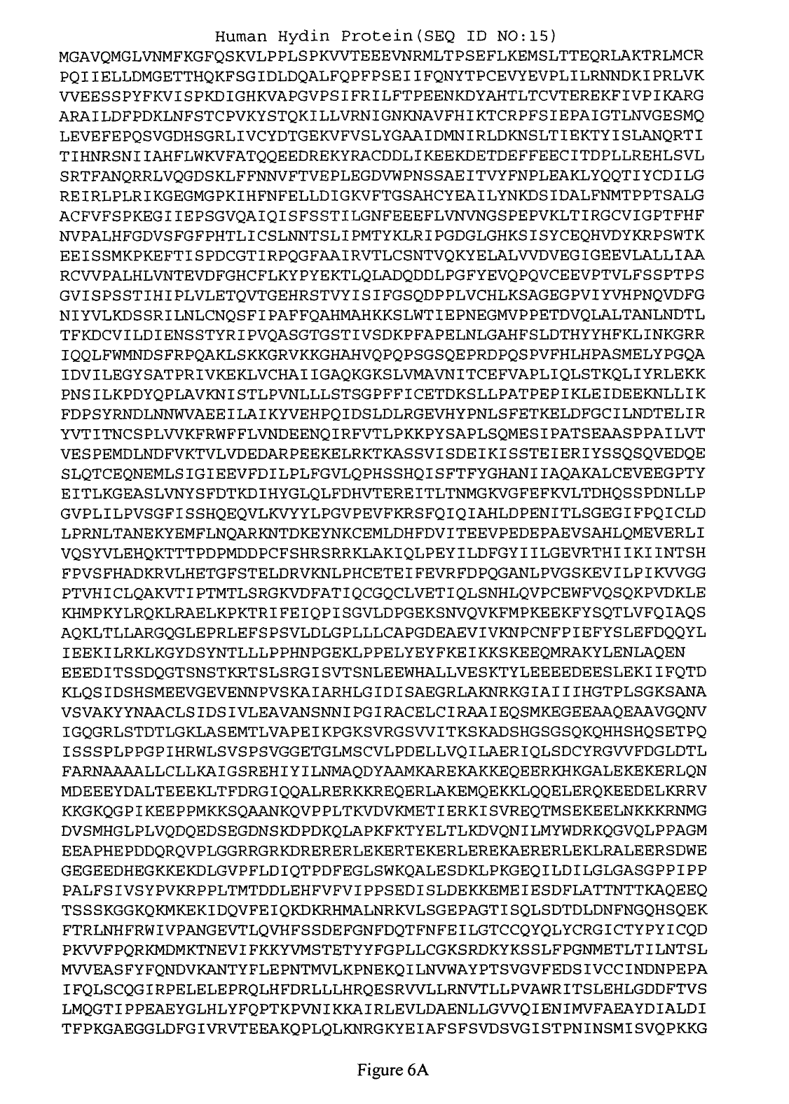

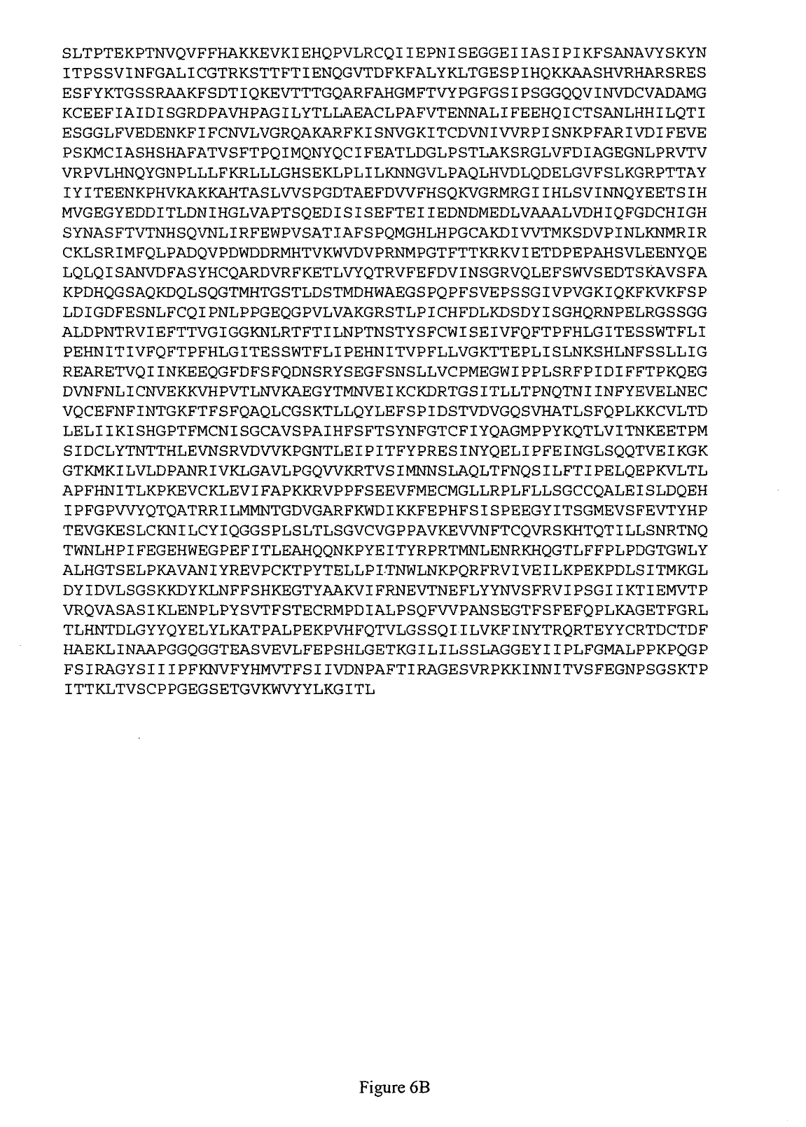

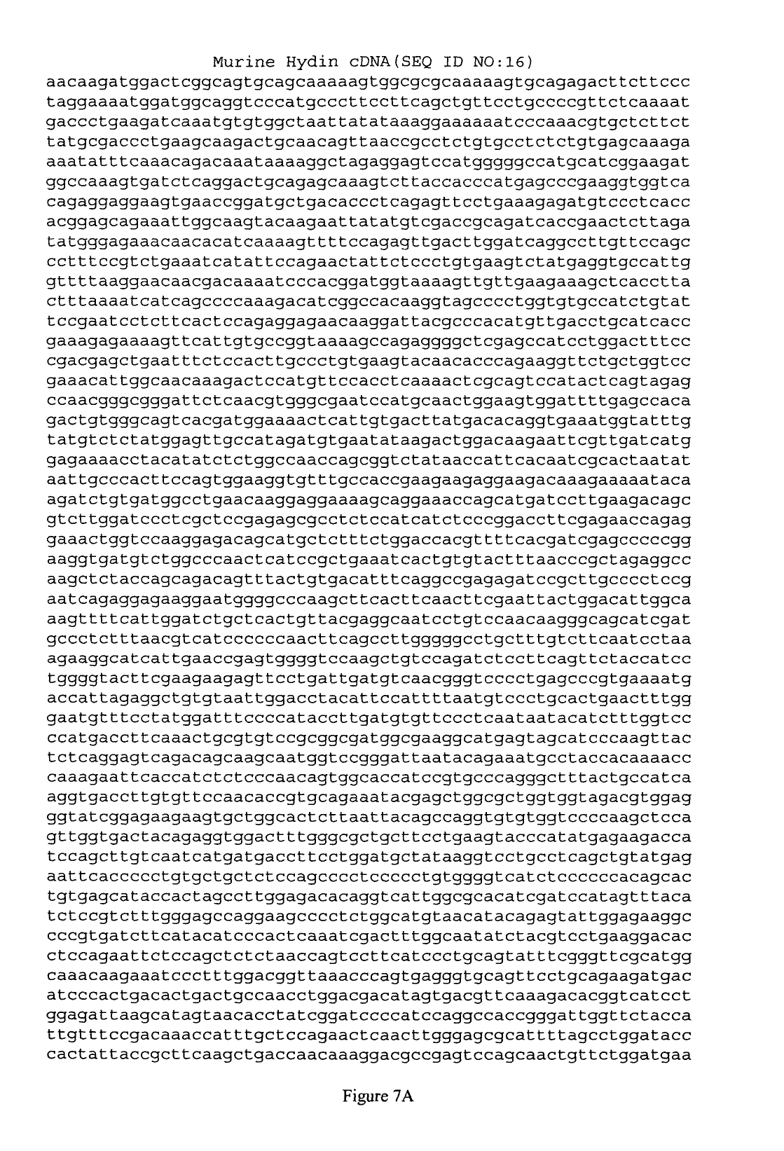

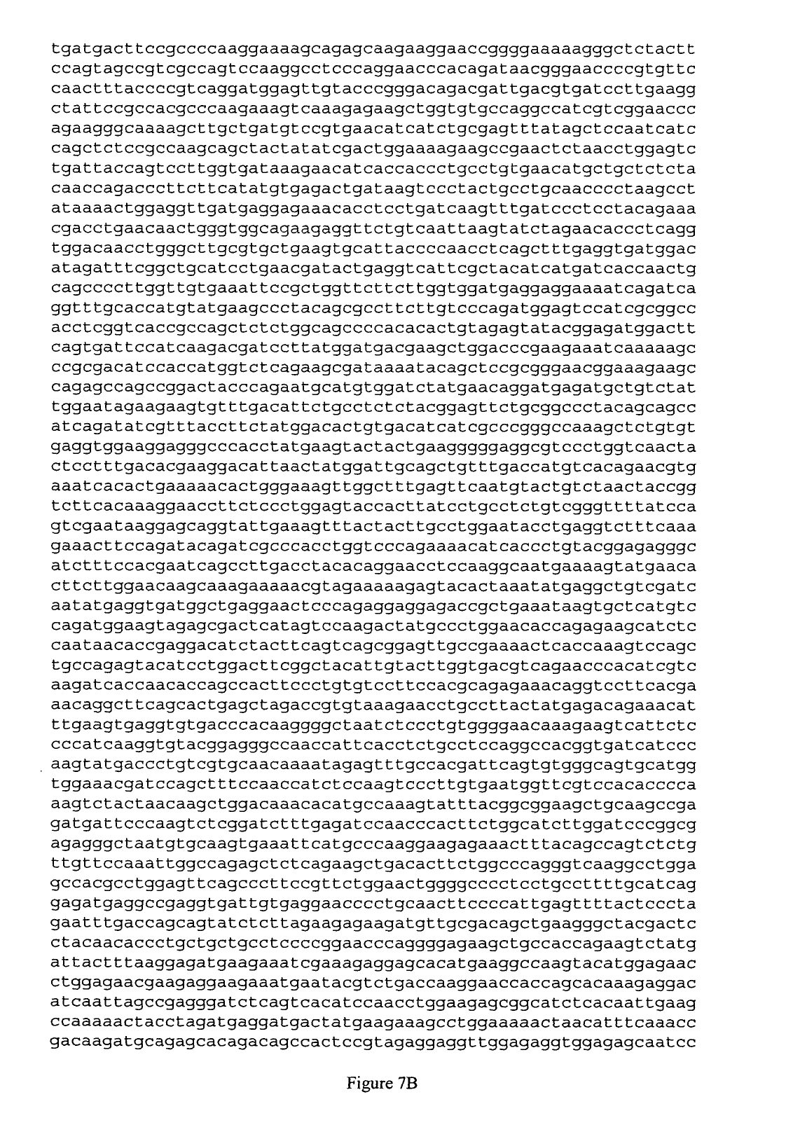

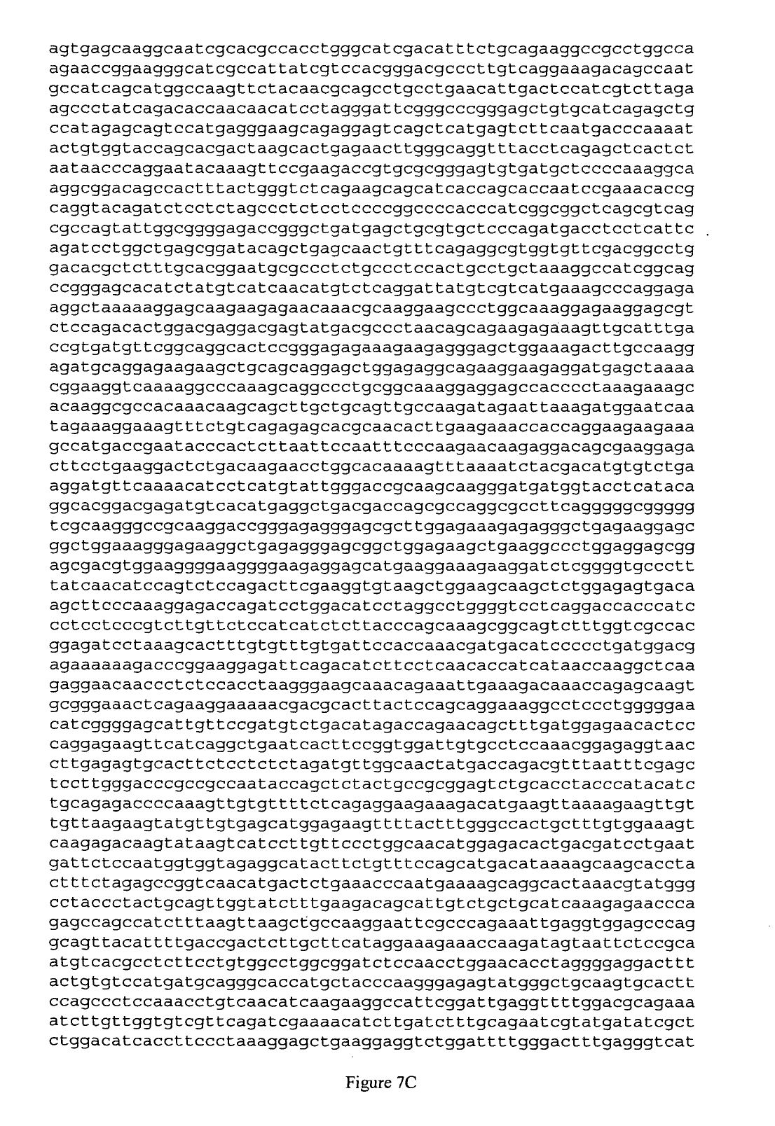

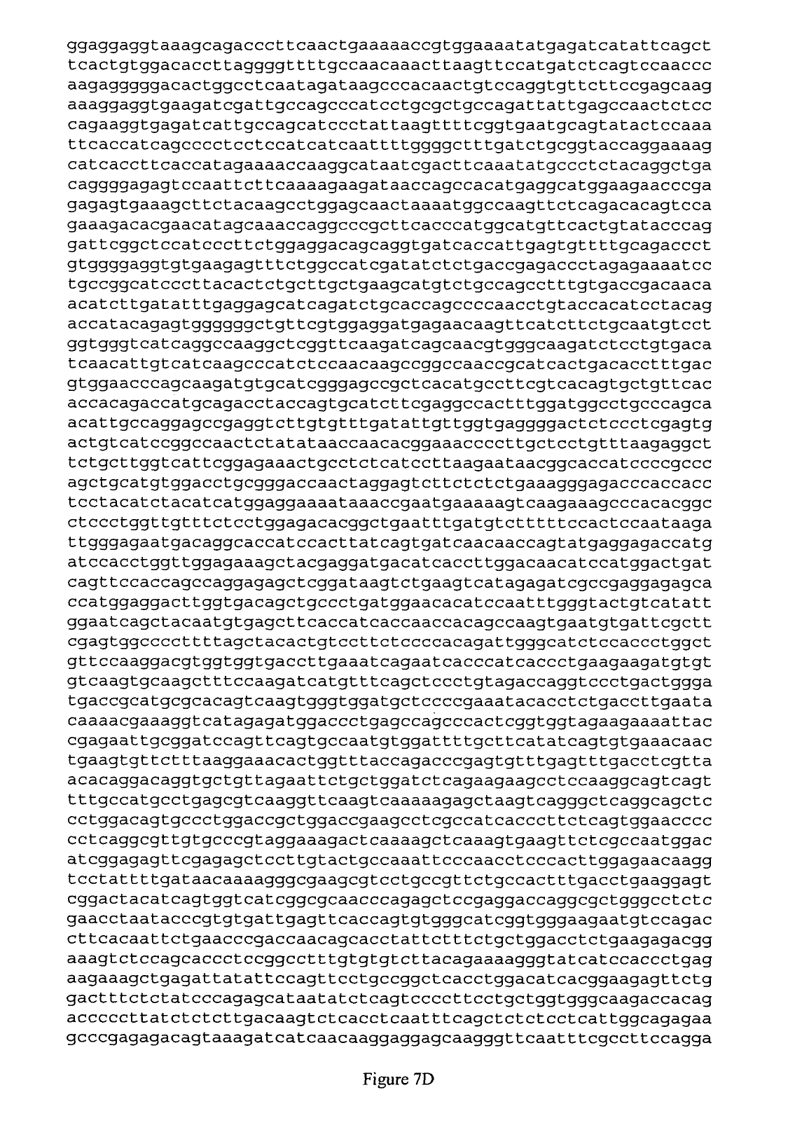

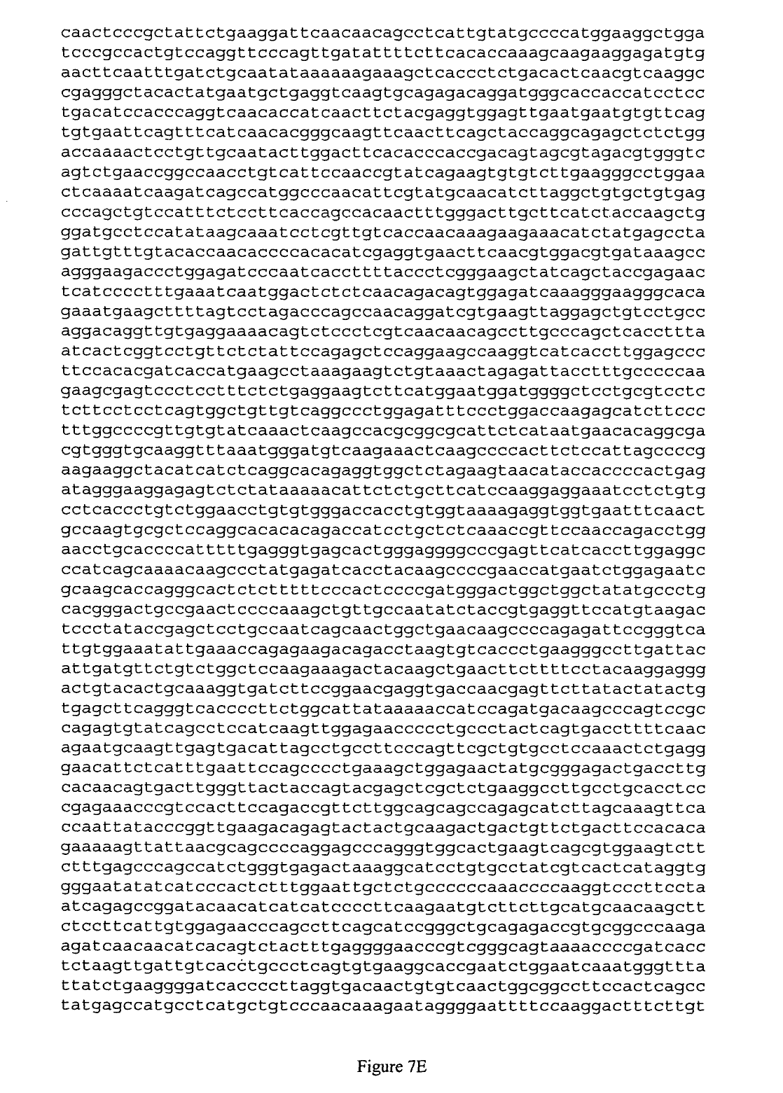

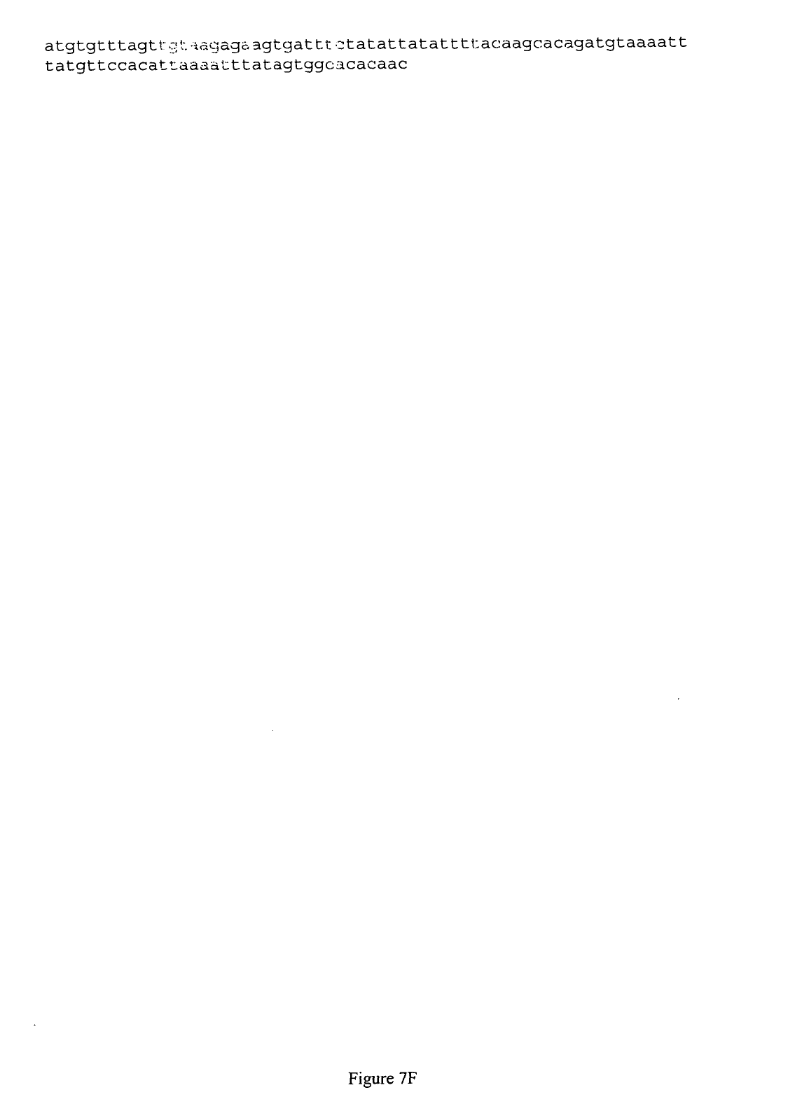

The cloning and sequencing techniques described in Example 6 were used to identify the human homologue from human testis RNA. The predicted full length human Hydin cDNA is set out as SEQ ID NO: 14 (FIG. 5). Currently, 95.5% of the predicted sequence has been confirmed by sequence analysis. The confirmed nucleotides of SEQ ID NO: 14 are as follows: nucleotides 141-7748; nucleotide 7929-14721; nucleotides 14777-15394. Northern blot analysis on RNA isolated from human testis detected expression of the human Hydin gene transcript that was 15.7 Kb. The predicted human Hydin polypeptide sequence is set out as SEQ ID NO: 15 (FIG. 6).

The present invention contemplates using the human Hydin gene and human Hydin polypeptide in a same or similar manner as described for the mouse Hydin sequences.

What is claimed:

1. An isolated polynucleotide comprising the nucleic acid sequence selected from the group consisting of:
(a) the polynucleotide sequence of SEQ ID NO: 1,
(b) the polynucleotide sequence of SEQ ID NO: 3.
(c) the polynucleotide sequence of SEQ ID NO: 14;
(d) the polynucleotide sequence of SEQ ID NO: 16;
(e) a polynucleotide that hybridizes under the following stringent conditions to the complement of any one of (a)-(e);
(1) hybridization at 65° C. in a hybridization buffer comprising 0.5 M NaHPO₄, and
(2) washing at 65° C. in a wash solution comprising 1×SSC.

2. An isolated polypeptide encoded by the polynucleotide of claim 1.

3. An isolated polypeptide of claim 2, wherein the polypeptide comprises the amino acid sequence of SEQ ID NO: 2.

4. An isolated polypeptide of claim 2, wherein the polypeptide comprises the amino acid sequence of SEQ ID NO: 15.

5. An antibody that specifically bind a polypeptide of claim 2.

6. A composition comprising the polynucleotide of claim 1 and a carrier.

7. A composition comprising the polypeptide of claim 2 and a carrier.

8. A method of detecting the Hydin gene comprising steps of:
(a) contacting a biological sample with a compound that binds to the polynucleotide of claim 1; and
(b) detecting binding between the compound and the polynucleotide,
wherein binding indicates the presence of the Hydin gene in the sample.

9. A method of detecting the Hydin polypeptide comprising steps of,
(a) contacting a biological sample with a compound that binds to the polypeptide encoded by the polynucleotide of claim 1; and
(b) detecting binding between the compound and the polypeptide,
wherein binding indicates the presence of the Hydin polypeptide in the sample.

10. The method of claim 9, wherein the compound that binds the polypeptide is an antibody,

11. A method of detecting a mutation in the human Hydin gene comprising steps of:
(a) contacting a biological sample with a compound that binds to the polynucleotide having the nucleic acid sequence of SEQ ID NO: 14; and
(b) detecting binding between the compound and the polynucleotide, wherein binding indicates the presence of a mutation in the human Hydin gene in the sample.

12. The method of claim 11, wherein the mutation is located at a position that corresponds to the position of the OVE459 mutation within the murine Hydin gene.

13. A method of diagnosing hydrocephalus in a human comprising detecting a mutation in the Hydin gene according to the method of claim 11; wherein the presence of the mutation in the human Hydin gene indicates a probability of the human developing hydrocephalus.

14. The method of claim 13, wherein the mutation is located at a position that corresponds to the position of the OVE459 mutation within the murine Hydin gene.

15. The method of claim 14, wherein the mutation is detected in a prenatal human.

16. A method of diagnosing a cilia dysfunction-related disorder comprising detecting a mutation in the Hydin gene according to the method of claim 11; wherein the presence of the mutation in the Hydin gene indicates a probability of the developing a ciliary dysfunction-related disorder.

17. The method of claim 16, wherein the cilia related disorder is selected from the group consisting of Kartagerner syndrome, primary cilia dyskinesia, chronic sinusitis, male infertility, deafness or kidney failure.
